(19) United States
(12) Patent Application Publication
(10) Pub. No.: US 2008/0281528 A1
(43) Pub. Date: Nov. 13, 2008

(54) System and Method for Environmental Sampling and Diagnostic Evaluation

(76) Inventor: Louis J. Relle Jr, Belle Chase, LA (US)

Correspondence Address: CARVER, DARDEN, DORETZKY, TESSIER, FINN,;BLOSSMAN & AREAUX, LLC, 1100 POYDRAS STREET, SUITE 3100, NEW ORLEANS, LA, 70163, US

(21) Appl. No.: 11/997,495

(22) PCT Filed: Aug. 3, 2006
(86) PCT No.: PCT/US06/30493

§ 371 Date: Jan. 31, 2008

Related U.S. Application Data
(60) Provisional application No. 60/705,384, filed on Aug. 3, 2005.

(51) Int. Cl. G01N 33/48

(52) U.S. Cl. 702/19; 702/24; 702/50; 73/31.07

(57) ABSTRACT

An environmental sampling system and method that collects biological contaminates in a removable cassette device via a sample capture media, such as a filter. The cassette device which is designed to specifically cause mixing of the air stream in a manner to get even distribution of the airborne particulates on the filter media has affixed thereto a data storage unit for storing other environmental parameters sensed and measured by the system in addition to the IAQ questionnaire information inputted by the investigator. After the sample period is complete, the system can be re-deployed in the field or environment by simply replacing the removable cassette device having the data storage unit. The collected biological contaminates in a removable cassette device and stored data in the affixed data storage unit are sent to a laboratory for analysis. The environmental sampling system may be operated independently of the removable cassette device to obtain and data log physical environmental data such as temperature, relative humidity, wind velocities, pressure differentials, and particulate counts.

### CROSS-REFERENCE TO RELATED APPLICATIONS

This application claims the benefit of earlier filed provisional patent application Ser. No. 60/705,384 and filed Aug. 3, 2005 which is incorporated herein by reference as if set forth in full below.

### BACKGROUND OF THE INVENTION

1. Field of the Invention

The present invention relates to indoor air quality environmental sampling of air particulates and related diagnostic evaluation. More particularly, to an environmental sampling system that includes a removable filter cassette device having a data storage unit affixed thereto (sometimes referred to herein as a “removable filter cassette device” or a “smart sample”) wherein, after environmental sampling of air particulates, the smart sample is sent to a laboratory so that both the environmental samples of air particulates, which may contain biological contamination, in the filter or other capture media, can be analyzed. The equipment operations and environmental physical parameters simultaneously captured during the sampling period can be down loaded from the data storage unit (which is a part of the smart sample) for evaluation.

2. General Background

Environmental samples of indoor air quality (whether in residences, commercial buildings, industrial settings, transport vehicles (airplanes, buses, trains, boats, etc.) or otherwise and whether from HVAC systems or from rooms or other enclosed spaces), as well as of outdoor air quality (taken as reference background samples), are obtained and used for a variety of commercial, health, regulatory and related legal purposes. Examples include, but are not limited to, determining: (a) whether, or to what extent, the air quality of an enclosed working area complies with a regulatory framework; (b) the affects of water or other moisture intrusion event in a residence (whether for determining insurance coverage or for other purposes); (c) whether, or to what extent, a landlord has breached its lease with a tenant due to problematic air quality; or, (d) whether, or to what extent, the vendor who was engaged by a homeowner to clean the HVAC ducts can show a material difference in the HVAC systems after cleaning.

An industrial hygienist or other specialist is often tasked with developing a means and method for obtaining such environmental samples in a manner that can be validated. Validation issues arise due to differences and inconsistencies in sampling techniques and methodologies, including, in some cases, the absence of related reliable and correlated environmental data. Also, when using impactors (such as spore traps or petri dishes) to sample, the samples do not lead to satisfactory results due to high variability and the inability to achieve statistically repeatable results (some of which is due to occlusion problems when samples are taken with the impactors for more than very short periods of time—e.g., when taken for more than five or ten minutes, depending on air stream quality). The use of filters as a sampling media can lead to more satisfactory results, but I am not aware of any cost-effective or convenient means that permit the use of filters as a sample media in sampling in HVAC systems over much longer periods of time (e.g., greater than 8 hours, including 24 hour/day cycles as well as multi-day tests). Logistical issues (e.g, the number of times that a probe needs to be reinserted in an air duct over long periods of time) as well as increased labor costs to attend to or operate the devices which are sampling, prohibit cost-effective statistically repeatable sampling techniques with filters over long periods of time.

Also, the number and duration of environmental samples is often ad-hoc and arbitrary (for example, five samples taken at various arbitrary times, each for an arbitrary interval of five minutes), although there are some general guidelines in the industry and some equipment manufacturers provide some information for using the manufacturer's equipment. In any event, given the present state of the industry, sampling does not generally occur over a long period of time. Samples taken over longer periods will result in better, stable and more valid, environmental samples (e.g., better standard deviations arise with samples taken over five minutes as opposed to one minute). In general, additional and/or longer environmental samples are not obtained due to the additional labor costs associated with the additional time required by the industrial hygienist to obtain the additional samples over the longer period of time as well as, in some cases (and, in particular with impactor-type sampling), the additional media and laboratory costs for the additional samples tend to prohibit cost-effective sampling.

Also, existing sampling methods do not necessarily distinguish or anticipate biological reproductive cycles or changes in such reproductive cycles. I speculate that is it very easy for a short time, or ad-hoc sample, to miss, a reproductive cycle. Also, existing sampling methods do not reliably account for seasonal issues (such as heating in the cooler months or cooling in the hotter months or windows, doors or other apertures being opened more often during certain months, such as in the spring or fall) that affect environmental samples.

Also, if associated environmental data (such as temperature, humidity, air speed, air volume, particle counts, pressure differential, HVAC cycle times, time of day, time of year or other relevant data) is recorded (either manually or with the help of instrumentation), such data must be correlated by, or for, the industrial hygienist and is not necessarily synchronized, or fully synchronized, with the samples. I speculate that there would be an advantage to having all of the data in a convenient database (e.g., so that graphs could be produced and overlaid as part of the analysis). Occupant loading of a building or other enclosed space, as well as access to and from such space may hold clues to the analysis (e.g., an increase in differential pressure may suggest that a local exhaust system was energized, such as a bathroom exhaust or kitchen range exhaust, or a drop in differential pressure levels may correlate with a window being opened. These two changes in differential pressure levels may account for significant changes in the relevant microbiological populations).

Further, there is no simple, convenient, cost-effective or automated means of capturing and delivering with the environmental sample (to a lab engaged to analyze the environmental sample) a set of correlated environmental data associated with the environmental sample (especially for samples taken over long periods of time—where “long periods” could be anything greater than 2 hours or where samples must be taken at several places in a building or other space). Further, existing manual methods of recording the associated environmental data are prone to human error (e.g., not taking timely notes of conditions or incorrectly identifying or mixing samples and such other associated environmental data or administrative data—e.g., room ids).

What I believe is needed, in particular, is at least the following:
1. a new way to address indoor air quality;
2. a simple automated means of capturing, and delivering, with the environmental sample (to a lab engaged to analyze the environmental sample) a set of environmental data associated with the environmental sample;
3. a means of achieving long sample times without an investigator or other operator having to continuously operate the equipment or otherwise be continuously on-site;
4. a set of environmental data to be delivered with the environmental sample wherein the set of environmental data is correlated with the environmental sample, is robust and was obtained over long periods of time; and, in a preferred embodiment, is self-correlated;
5. a better way of analyzing the affects of water or other moisture intrusion events in indoor settings;
6. a system and method of environmental sampling that enjoys a high level of integrity so as to withstand legal and judicial scrutiny;
7. a more reliable method of benchmarking the air quality of indoor or outdoor areas prior to the initiation of construction, remedial or other activities for comparison with later air quality samples taken during, and/or upon completion of, said construction, remedial or other activities;
8. a simple and inexpensive means of building a master database of typical or normalized air quality conditions in residences, buildings, transport vehicles and other commercial or industrial spaces; and to use said master database to set norms for air quality conditions in said residences, buildings, transport vehicles and other commercial and industrial spaces;
9. an efficient and standard data log or other database format for capturing environmental data associated with an environmental sample;
10. a means for insuring that certain information is entered into an environmental sampling system by an operator prior to operating the system.
11. The data storage unit for each filter cassette will also be transporting information from an IAQ questionnaire relating to health complaints of the occupants, health diagnosis for the occupants, building science information and typical building use information to correlate with the environmental sample, and subsequently to be included in a data base. This data base will provide direct correlation with all aspects of the sample with the environmental results and the IAQ questionnaire information, allowing analysis of trends and human biological response levels to various levels of particulate contamination specific to the type of contamination.

As will be seen more fully below, the present invention is substantially different in structure, methodology and approach from that of other environmental sampling and diagnostic evaluation systems.

The predominant sampling technique used presently in the indoor air quality arena is what is generally termed as a spore trap. A spore trap uses the technology of impaction to capture airborne spores. Spore traps have an inlet and outlet to a plastic cassette with a small plastic rectangular plate which has a sticky substance on one side as the capture media. The cassette inlet is a slot which accelerates the air stream at the sticky side of the plastic rectangular plate where the physics of inertia will cause the spores in the air stream to be impacted into the sticky substance. Manufacturers of the spore traps and other scientists have published test data suggesting that these sport traps have a capture efficiency of approximately 70% of the smaller spore sizes and the larger spores impact with enough energy to cause a phenomena of bounce, therefore many of the larger spores are not caught by the spore trap. U.S. Pat. No. 5,437,198, U.S. Pat. No. 4,764,186, U.S. Pat. No. 5,693,895 describes impaction devices which would be classified as spore traps in the indoor air quality industry. These impactors are limited to sample periods of 30 minutes or less due to the amount of air required to obtain the proper velocity in the cassette to cause the capture of the 1 micron to 15 micron spores. It is know that this relatively high sample volume over a relatively long time will cause occlusion of the sample and not allow microscopic analysis.

A second sampling method which has been used for many years by industrial hygienists to collect airborne particulates is a filter cassette with an inlet and outlet. The air is drawn through the inlet where it is forced to pass through a filter media with specific pore sizes and through a backer support pad and then out the outlet. The air is drawn through the filter cassette using a calibrated vacuum pump for a specified time. The air flow rate through the filter cassette is typically much lower than the impactors, typically in the 0.1 liter per minute to 5 liter per minute flow rates. Existing filter cassettes range from 10 millimeters to 47 millimeters in diameter for industrial hygiene use. There are much larger filter sizes used for environmental use for what is termed PM 10 and PM 2.5 sampling to determine emissions for compliance with U.S. Environmental Protection Agency clean air standards. The draw back with using the existing filter cassettes for microscopic fungal analysis is the time required for the mycologist to analyze one sample. I speculate that the mycologist would not be able to effectively read a single 25 millimeter diameter filter in a normal eight hour work day. The typical mycologist can read a spore trap trace size of 2 millimeters by 14 millimeters in 15 to 20 minutes. The increase cost of using full sized filters for fungal analysis in indoor air quality investigations make this method cost prohibitive. Using the time for analysis as a basis of cost, the filter analysis would be 24 times greater than the present cost for each sample. To address this issue, U.S. Pat. No. 6,779,411 appears to me to use restrictor plates with a 25 millimeter diameter filter to minimize the deposition area of the filter and allow the mycologist to analyze the filter in a much shorter time frame, therefore keeping the cost to a level which is reasonable. I speculate that a drawback to using the filters as described in U.S. Pat. No. 6,779,411 is the significant vacuum pressure needed to draw the air through the sample collection media and the small area causes the samples to become too occluded for microscopic analysis after sampling periods of typically less than 10 hours.

### SUMMARY OF THE PRESENT INVENTION

The preferred embodiment of environmental sampling and diagnostic evaluation system of the present invention solves the aforementioned problems in a straight forward and simple manner.

Broadly, the present invention contemplates an environmental sampling system of air for air particulates comprising: a means for sampling the air to create an air sample; a means for capturing from the air sample, a sample of the air particulates in the air sample; a means for storing the air particulates sample in a removable cassette device; a means of accumulating and data logging building science information, human health related information of building occupants, building use information specific to the environmental sample and, a means for sampling a plurality of environmental physical parameters associated with the air sample during or surrounding a sampling period during which the air sample was sampled, wherein the removable cassette device has a filter or other media for capturing and storing the air particulates sample, including biological particulates, and a data storage unit affixed thereto for storing samples of said plurality of environmental physical parameters and wherein, after the sampling period, the removable cassette device (a/k/a the smart sample) is sent to a laboratory so that both the air particulates sample, which may contain biological contamination, in the filter media or other capture media in the removable cassette device can be determined and the stored environmental physical parameters and building use/occupant/science information obtained from the IAQ questionnaire can be downloaded from the data storage unit for evaluation with the air particulates sample.

An object of the present invention is to provide an environmental sampling system that can be advantageously used to improve indoor air quality.

Another object of the present invention is to provide an environmental sampling system that improves air quality analysis.

A further object of the present invention is to provide an environmental sampling system that improves the analysis of airborne biological particulates in an environment.

A still further object of the present invention is to provide an environmental sampling system that is flexible and that can be installed in a variety of modes in a duct cavity of a HVAC system in a residence, commercial building, or transport vehicle, that can be used in other indoor areas or that can be used outdoors.

A still further object of the present invention is to provide an environmental sampling system that includes an environmental sampling/sensor probe assembly adapted to be mounted in a duct cavity of a HVAC system, such as in the ceiling or wall, and a remote control assembly for sampling additional environmental parameters and controlling the environmental sampling/sensor probe assembly.

A still further object of the present invention is to provide an environmental sampling system that includes an environmental sampling/sensor probe assembly having a removable cassette device, such removable cassette device housing a sample capture media such as, without limitation, a filter and a data storage unit for storing data from sensors and monitors.

A still further object of the present invention is to provide an environmental sampling system that can operate over long periods of time (e.g., about 8 hours or for several days) without the need for an investigator to “man” or otherwise operate the environmental sampling system or to be on-site continuously during such sampling. This environmental sample may be analyzed by either microscopy or other analytical methods.

In view of the above objects, it is a feature and/or advantage of the present invention to provide:
1. an environmental sampling system that is easy to install and use;
2. an environmental sampling system that is relatively simple structurally and is compact;
3. an environmental sampling system that collects biological contaminates or other biological particulates in a removable/replaceable cassette device having a data storage unit for storing related IAQ questionnaire information and environmental parameters;
4. an environmental sampling system that collects biological contaminates or other biological particulates in a removable/replaceable cassette device having a data storage unit for storing related IAQ questionnaire information and environmental parameters and for storing environmental data collected while said biological contaminates or other biological particulates were collected;
5. an environmental sampling system that collects biological contaminates or other biological particulates in a removable/replaceable cassette device having a data storage unit for storing related IAQ questionnaire information and environmental parameters, for storing environmental data collected while said biological contaminates or other biological particulates were collected and, for storing programming testing sequences for testing the environmental parameters
6. an environmental sampling system that can be re-deployed in the field or environment by simply replacing the removable/replaceable cassette device having a data storage unit;
7. an environmental sampling system that can sample ambient air in many environments;
8. an environmental sampling system that samples air in an air-duct cavity of an HVAC system;
9. a simple automated means of capturing and delivering, with the environmental sample (to a lab engaged to analyze the environmental sample) a set of environmental data and IAQ questionnaire information associated with the environmental sample;
10. a set of environmental data to be delivered with the environmental sample wherein the set of environmental data is correlated with the environmental sample, is robust and was obtained over long periods of time such as, without limitation, durations of 5 minutes to 24 hours or longer; and, in a preferred embodiment, is self-correlated;
11. a better way of analyzing the effects of water or other moisture intrusion events in indoor settings such as to create a baseline, by sampling immediately after an event, and to return to the same environment after clean up to further sample and determine the environmental changes;
12. a system and method of environmental sampling that enjoys a high level of integrity so as to withstand legal and judicial scrutiny;
13. a more reliable method of benchmarking the air quality of indoor or outdoor areas prior to the initiation of construction, remedial or other activities for comparison with later air quality samples taken during, and/or upon completion of, said construction, remedial or other activities;
14. a simple and inexpensive means of building a master database of typical or normalized air quality conditions in residences, buildings, transport vehicles and other commercial or industrial spaces; and to use said master database to set norms for air quality conditions in said residences, buildings, transport vehicles and other commercial and industrial spaces; and,
15. an efficient and standard data log or other database format for capturing environmental data and IAQ questionnaire information associated with an environmental sample;
16. an environmental sampling system for an HVAC system that can automatically sample only when the HVAC system is circulating air;
17. an environmental sampling system for an HVAC system that can automatically sample only when the HVAC system is not circulating air;
18. an environmental sampling system for an HVAC system that can regulate the air stream through a sample filter in a removable cassette to match or exceed at a predetermined level the air stream through the HVAC system;
19. a means to compile collected air particulate samples to correlate causality of health conditions of habitants of a building, residence, industrial setting or transport vehicle to the environments and to remedy such causality by developing improvements to the indoor environment; and,
20. a means to deploy multiple environmental sampling units in a building, residence, or other enclosed place and to synchronize sampling amongst the units via wire or wireless communications.
21. The data storage unit for each filter cassette will also be transporting information from an IAQ questionnaire relating to health complaints of the occupants, health diagnosis for the occupants, building science information and typical building use information to correlate with the environmental sample, and subsequently to be included in a data base. This data base will provide direct correlation with all aspects of the sample with the environmental results and the IAQ questionnaire information, allowing analysis of trends and human biological response levels to various levels of particulate contamination specific to the type of contamination.

The above and other objects, features and advantages of the present invention will become apparent from the drawings, the description given herein, and the appended claims.

### BRIEF DESCRIPTION OF THE DRAWING

For a further understanding of the nature and objects of the present invention, reference should be had to the following description taken in conjunction with the accompanying drawings in which like parts are given like reference numerals and, wherein:

FIG. 1A illustrates a perspective view of the environmental sampling system in accordance with the present invention with the removable cassette device shown in cross-section;

FIG. 1B illustrates a perspective view of the environmental sampling system in accordance with FIG. 1A with the interior components of the vacuum pump and system control assembly shown in phantom;

FIG. 1C illustrates a perspective view of an alternate connection scheme of the environmental sampling system in accordance with FIG. 1A;

FIG. 2 illustrates a plan view of the vacuum pump and system control assembly of the environmental sampling system in accordance with the present invention with the housing removed;

FIG. 3 illustrates a plan view of the environmental sampling/sensor probe assembly of the environmental sampling system in accordance with the present invention;

FIG. 4A illustrates cross-sectional view of the removable cassette device (a three piece cassette with transportation plugs) in accordance with the present invention;

FIG. 4BA illustrates a perspective view of the cassette inlet section (Part C) of the FIG. 4A;

FIG. 4BB illustrates an end view of the environmental filter cassette inlet section of FIG. 4BA;

FIG. 4BC illustrates a cross-sectional view along the plane A-A of FIG. 4BB;

FIG. 4BD illustrates a cross-sectional view along the plane B-B of FIG. 4BB depicting the mixing chamber;

FIG. 4BE illustrates a cross-sectional view along the plane D-D of FIG. 4BD;

FIG. 4CA illustrates an end view of the cassette funnel of FIG. 4A to trace section with trace opening and quarter/half marking pins;

FIG. 4CB illustrates a cross-sectional view along the plane A-A of FIG. 4CA;

FIG. 4CC illustrates a cross-sectional view along the plane C-C of FIG. 4CA;

FIG. 4CD illustrates detail A of FIG. 4CC depicting a funnel ridge of the environmental filter cassette funnel to trace section with trace opening and quarter/half marking pins;

FIG. 4DA illustrates an end view of the cassette suction section of FIG. 4A with the taper from the trace opening to a circular suction (fitting) nipple;

FIG. 4DB illustrates a cross-sectional view along the plane A-A of FIG. 4DA;

FIG. 4DC illustrates a cross-sectional view along the plane B-B of FIG. 4DA;

FIG. 4E illustrates a peripheral particle counter of the environmental sampling system coupled to a cross-sectional view of the environmental sampling/sensor probe assembly;

FIG. 5 illustrates the duct air speed detector of the environmental sampling system of the present invention;

FIG. 6 illustrates a perspective view of the environmental sampling system installed for sampling a duct cavity of a HVAC system;

FIG. 7 illustrates the general block diagram of the control circuitry of the vacuum pump and system control assembly in accordance with the present invention;

FIG. 8 illustrates the general block diagram of the communications between the vacuum pump and system control assembly, the environmental sampling/sensor probe assembly and other sensors and detectors in accordance with the present invention;

FIG. 9A illustrates the general diagram of the data log of an environmental sample in accordance with the present invention;

FIG. 9B illustrates the general diagram of the data log of an environmental sample in accordance with the present invention for multiple sensors, detectors or systems;

FIG. 10 illustrates the flow chart of the environmental sampling operations in accordance with the present invention;

FIG. 11A illustrates a sample location with multiple areas being sampled;

FIG. 11B illustrates a sample location with multiple areas being sampled by a master with a plurality of slaves;

FIG. 12 illustrates an exemplary set of accessory sensor;

FIG. 13 illustrates system for laboratory analysis with program instructions;

FIG. 14 illustrates a laptop in communication with one or more of the environmental sampling systems;

FIG. 15A illustrates a master testing sequencing database with a variety of building science tests for use by the environmental sampling system; and,

FIG. 15B illustrates a master database of IAQs for a variety of building sciences for use by the environmental sampling system.

### DETAILED DESCRIPTION OF THE PREFERRED EMBODIMENT

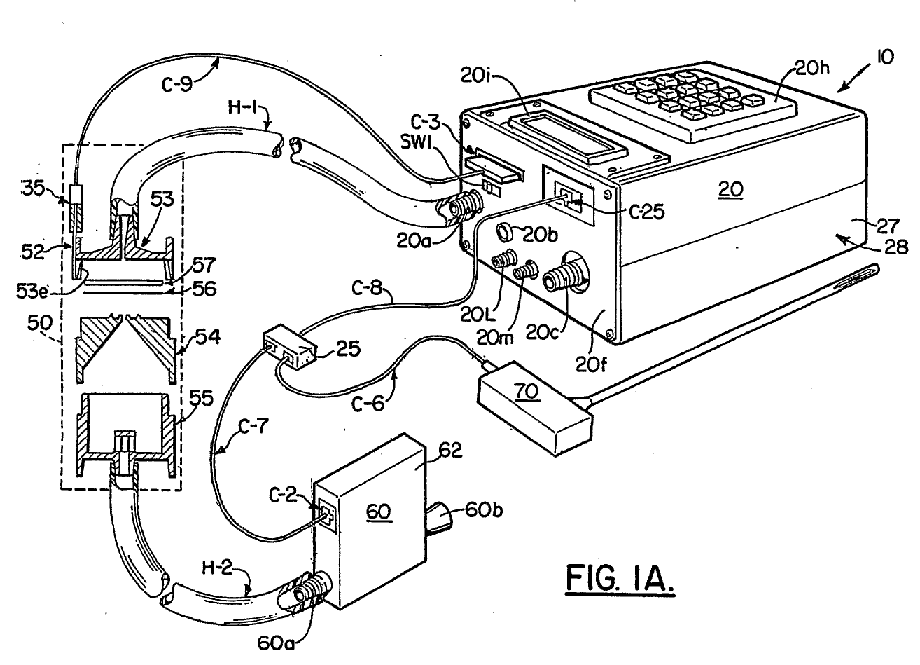

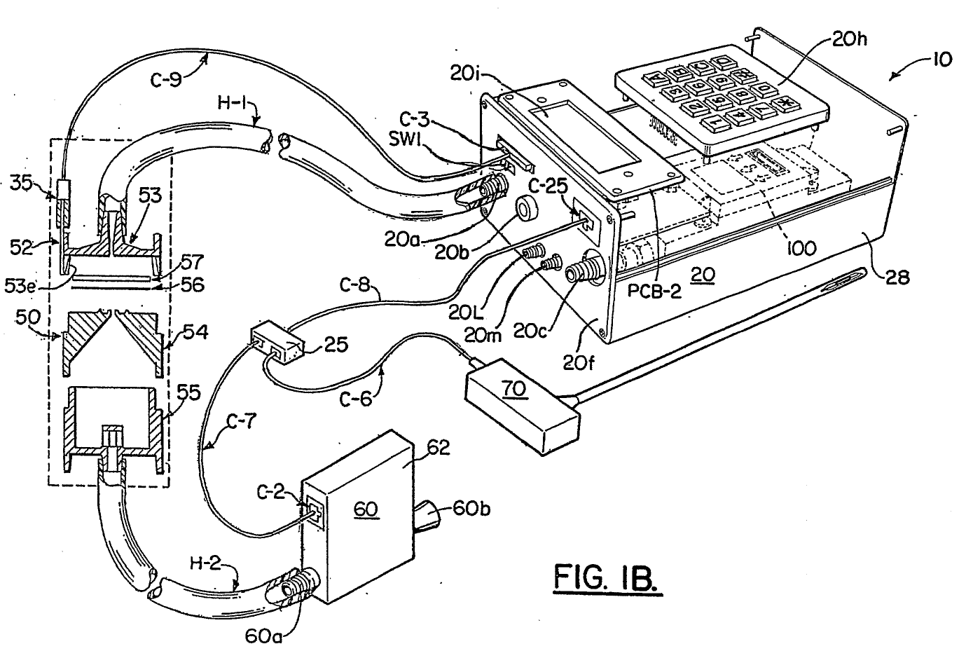

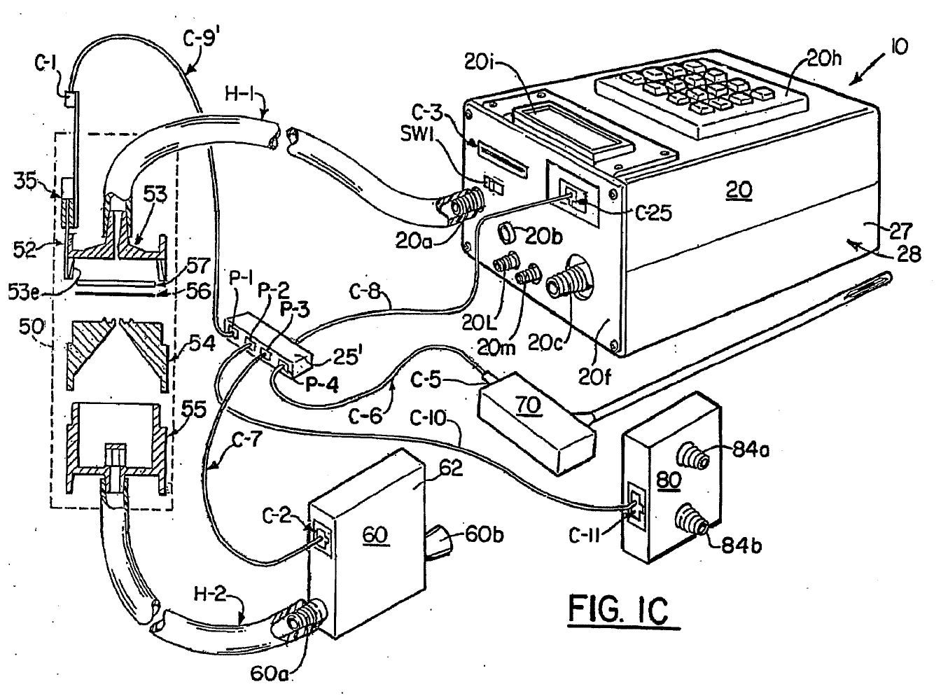

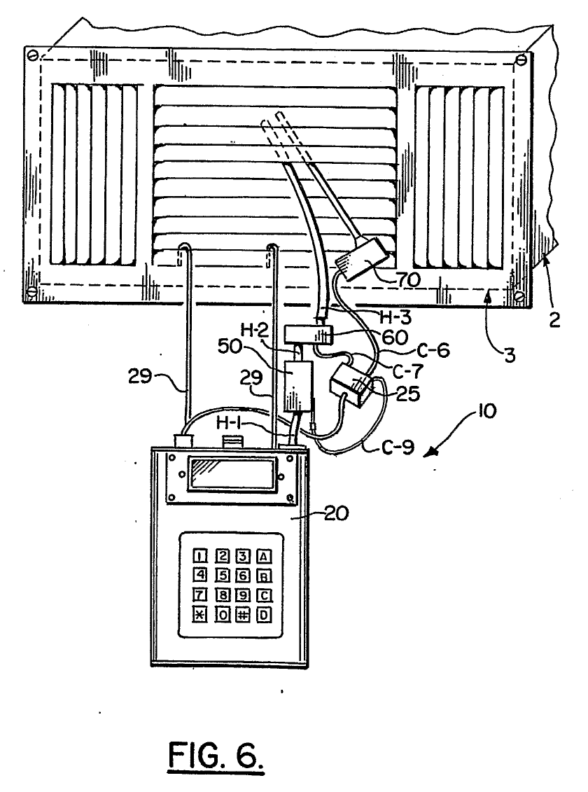

Referring now to the drawings and in particular FIGS. 1A, 1B, 2, 3, 4A and 5 the environmental sampling system of the present invention will generally be referenced by the numeral 10. The environmental sampling system 10 includes, in general, a vacuum pump and system control assembly 20 and an environmental sampling/sensor probe assembly 50 adapted to be mounted in or an air duct 2 of a Heating Ventilation and Air Condition (HVAC) system (FIG. 6) installed in a residence, office building, transport vehicle (whether airplane, bus, train or otherwise) or the like. As will be seen from the description given below the environmental sampling/sensor probe assembly 50 includes a removable cassette device 51 that is removed, after the sampling period is complete, and sent to a laboratory for evaluation. The removable cassette device 51 is easily replaced so that the environmental sampling/sensor probe assembly 50 can be immediately re-used in the field. The removable cassette device 51 with attached data storage unit 52 can be replaced in the field by simply attaching a vacuum hose H1 to the suction (fitting) nipple 53a and coupling the data storage unit 52 to a suitable card holder 35 for reading and/or writing data to the data storage unit 52. The card holder 35 is in electrical communication with the microprocessor 152 via external connection interfacing 153. The removable cassette device 51 is also attached to particle counter 60 in the field by simply attaching hose H2 to the inlet (fitting) nipple 55a.

In the exemplary embodiment, the data storage unit 52 can be one of a data storage card, memory flash card or smart card operable to send and receive data for downloading and storing therein. Other computer readable medium may be substituted such as integrated chips, flash disk sticks with universal serial bus (USB) connection can be also used. The card holder 35 would be substituted with the appropriate connection.

In FIG. 1A, the card holder 35 is shown attached to the vacuum pump and system control assembly 20 via cable C9 to connector C3. Alternately, in FIG. 1C, the card holder 35 is shown attached to splitter 25′ via cable C-9′. The data storage unit 52 is shown connected to card holder 35.

Referring still to FIG. 1A, the splitter 25 serves as a multi-port connection strip with two ports. The output of which is coupled to cable C8 to cable connection C25. In FIG. 1C, the component defining splitter 25′ serves as a multi-port connection strip with multiple ports P1, P2, P3 and P4 coupled to cable connection C25 integrated into housing 28. Nevertheless, the multi-port connection strip (splitter 25) may include up to 32 ports to attach up to 32 sensors or detectors.

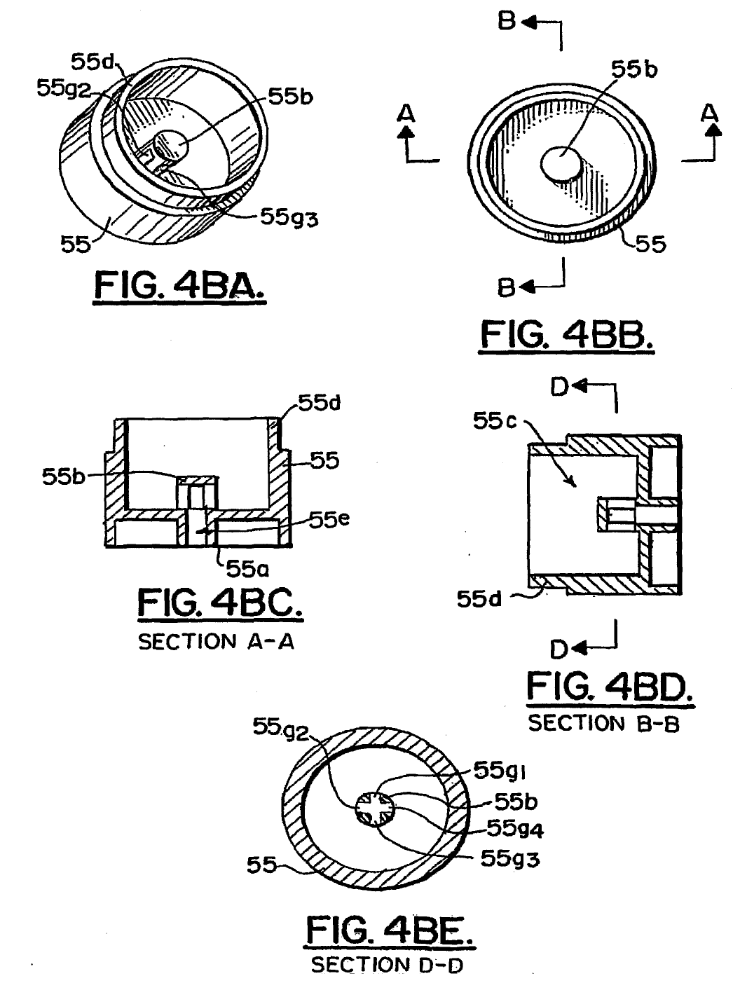

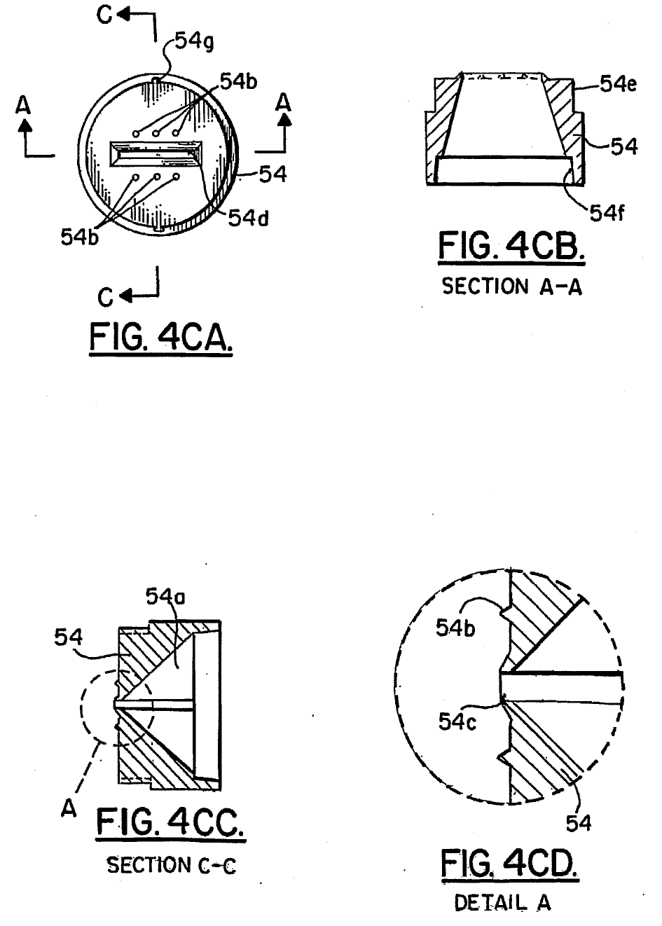

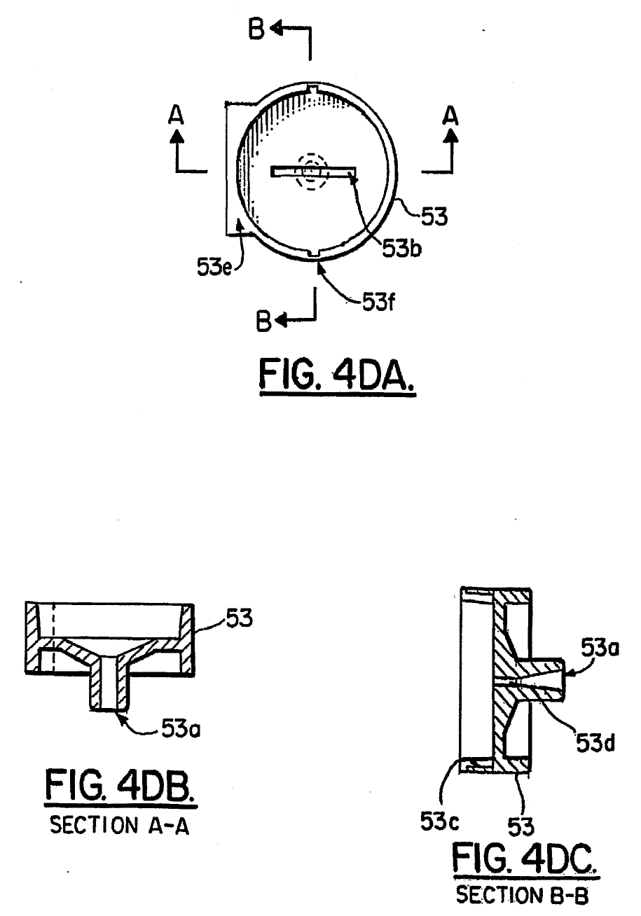

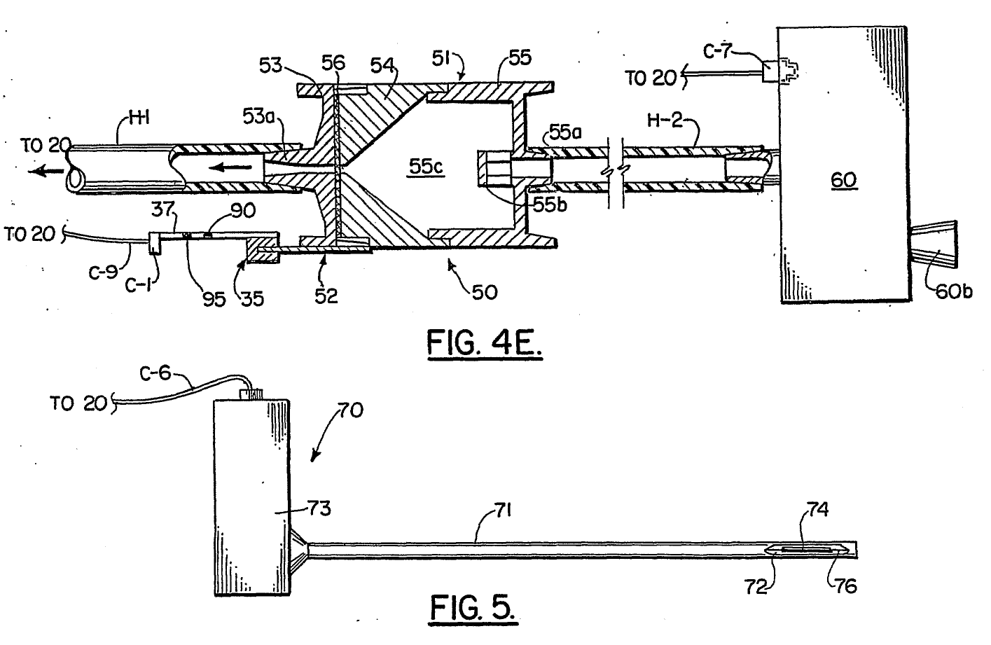

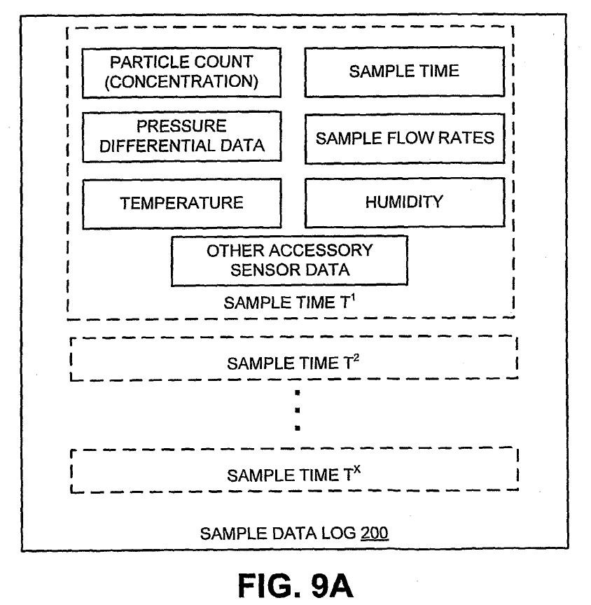

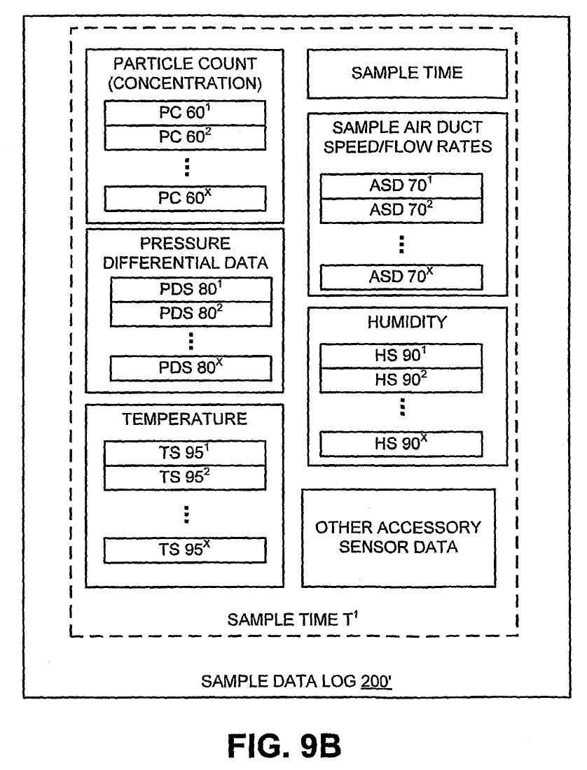

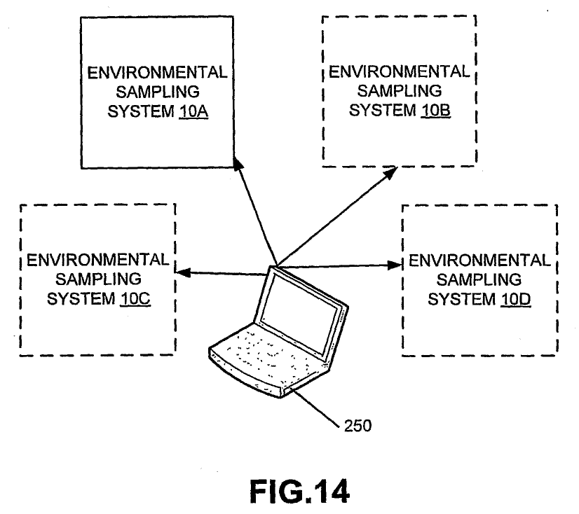

The environmental sampling/sensor probe assembly 50, includes a data storage unit 52, as best seen in FIG. 1A and FIGS. 4A, 4BA, 4BB, 4BC, 4BD, 4BE, 4CA, 4CB, 4CC, 4CD, 4DA, 4DB, and 4DC. The data in the data storage unit 52 is stored in a sample data log 200, as best seen in FIG. 9A. The data log 200 once created can be downloaded to a database file 312 at the laboratory or in the field by the investigator. An investigator (industrial hygienist) may also collect other information on health or other environmental issues from the habitants (such as, but not limited to, tracking data of illnesses associated with those habitants of the sampled environment) either by interviewing said habitants or providing them with a health and environmental survey to complete, or some combination thereof (said collection referred to herein as the “IAQ questionnaire information”). As best seen in FIG. 14, this IAQ questionnaire information may be entered by the investigator via laptop 250 and will be stored in IAQ questionnaire data log 202 of the data storage unit 52. I speculate that analysis of the IAQ questionnaire information, when combined with analysis of the captured air particulates in the removable cassette device 51 and the physical environmental data in the data storage unit 52, is likely to provide an enhanced indoor air quality analysis and lead to greater improvements in indoor air quality.

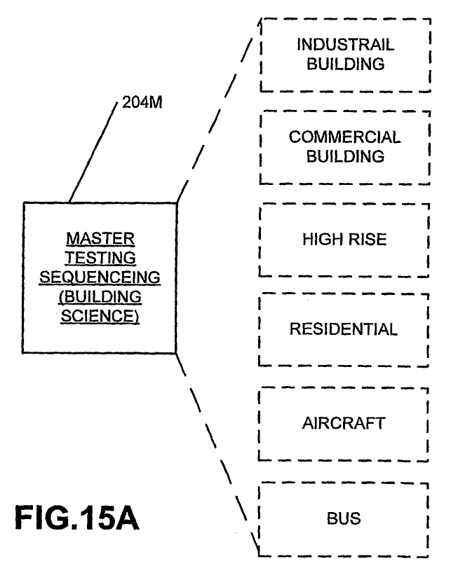

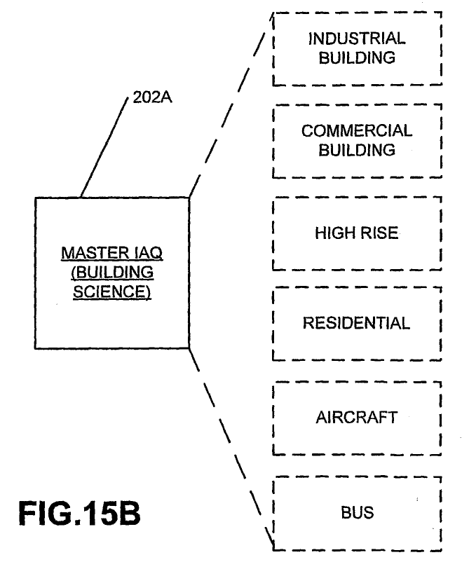

With specific reference also to FIG. 15A, programmed testing sequencing 204 is stored in the data storage unit 52 prior to testing or delivering the data storage unit 52 to the field. In the exemplary embodiment, depending on the building science a different set of testing instructions may be required. Thus, a master testing sequencing database 204M of the building science is used to program the data storage unit 52 with the designated testing sequence. For example, a different testing sequence may be required for an industrial building, commercial building, high rise, residential, aircraft, bus, boat etc.

Moreover, the programmed testing sequencing 204 for the building science may also be specific or related to a specific event such as without limitation after flooding, a fire, chemical release, or other exposure in the building.

With specific reference also to FIG. 15B, the IAQ for the test may be stored in a master IAQ database 202A for the building science and related testing. Thus, the master IAQ database 202A of the building science can be loaded into laptop 250 or the data storage unit 52. As the investigator enters the data via the laptop 250, the entered information is stored in the IAQ data log 202. Thus, a different IAQ may be required for an industrial building, commercial building, high rise, residential, aircraft, bus, boat etc.

Referring now to FIGS. 1A, 1C, 2, and 3, the environmental sampling/sensor probe assembly 50, the particle counter 60 and duct air speed detector 70 and remote (optional) pressure differential sensor 80 includes five power/communication cables C9′, C8, C7, C6, and C10, respectively, for connection to the splitter 25′. On end of the cables are connected at connections ports C1, C2, C5 and C11 of the card holder 35, the particle counter 60, the duct air speed detector 70 and the pressure differential sensor 80, respectively. The other end of the cables C9′, C7, C6, and C10, are connected to a respective one port in the splitter 25.

While the exemplary embodiment, describes includes five power/communication cables C9′, C8, C7, C6, and C10 one or more of these cables or other cables may be replaced with a wireless communication medium. In such a case, each sensor (such as, the particle counter 60, the duct air speed detector 70 and the pressure differential sensor 80, temperature sensor 95 and humidity sensor 90, accessory sensors 170) would include a wireless communication unit for communicating with the vacuum pump and system control assembly 20 and a source of power.

In the exemplary embodiment, each of the connectors C1 C2, C3, C4, C5, and C11 include a plurality of contacts wherein some of the contacts deliver power to the card holder 35, the particle counter 60, the duct air speed detector 70 and pressure differential sensor 80 while other contacts signal or otherwise communicate with the vacuum pump and system control assembly 20 via cables C6, C7, C8, C9 and C10.

As best seen in FIG. 1A, FIGS. 4A, 4BA, 4BB, 4BC, 4BD, 4BE, 4CA, 4CB, 4CC, 4CD, 4DA, 4DB, and 4DC the removable cassette device 51 includes a Part A 53 having a rounded end male suction (fitting) nipple 53a designed for a 5/16 inch inner diameter pneumatic suction hose H1 to be affixed, a tapered sample suction canal 53d transitioning from a 4.5 millimeter diameter opening on the exterior to a 2 millimeter by 22 millimeter rectangular suction opening 53b at the filter support surface designed to hold a 37 millimeter diameter filter backer pad 57 (shown in FIG. 1A) and 37 millimeter diameter filter 56 as shown in FIG. 1A. Part A 53 has a flattened exterior section 53e designed to have the data storage unit (SD card) 52 affixed. In the exemplary embodiment, the data storage unit 52 lies flush on the ledge or shelf created by the flattened exterior section 53e and can be affixed adhesively thereto. Nevertheless, the data storage unit 52 may be affixed, integrated or attached through other means.

Preferably, the data storage unit 52 is affixed to the removable cassette device 51 so that the data related to the biological sample are not misplaced or otherwise disassociated with the computer readable data stored and collected in the data storage unit 52. Hence, the integrity of all data is protected.

Part A 53 has an opening which tapers from 38 millimeters down to 37 millimeters in a manner to provide an air tight mating surface for attachment to Part B 54. This taper also has two alignment slots 54g which requires the suction opening/slot 53b to align with the trace slot 54d of Part B 54. Part B 54 having a trace slot 54d 2 millimeters wide by 22 millimeters long, aligns with the suction opening/slot 53b of Part A 53 when the two parts are placed together with a backer pad 57 and a filter 56 between the two parts. Part B 54 has a 1 millimeter high ridge 54c around the perimeter of the trace slot 54d. It is designed to make a visible indention in the filter 56 outlining the location of the trace on the filter 56. There are six raised pins 54b, 1 millimeter in height along the long side of the trace slot 54d, set as to divide the trace into four equidistant sections. These pins 54b will also leave visible indentions in the filter 56 allowing the analytical laboratory the ability to cut the filter trace in half or quarter sections. Part B 54 will have a tapered section with two alignment pins 54g to allow the perfect alignment of the suction opening/slot 53b and the trace slot 54d when the parts are put together. The tapered sections 54e and 53c are designed to mate and allow an air tight seal between Part A 53 and Part B 54 when put or mated together. Part B 54 has a funnel 54a section which directs the air flow from the mixing chamber 55c of Part C 55 without any flat areas which could support deposition of the particulates in the sampled air stream. The funnel 54a transitions from a 34 millimeter circle at its inlet to a 2 millimeter by 22 millimeter trace slot 54d at the point where the filter 56 is located. There will also be a tapered sections 54f and 55d designed to mate and allow an air tight seal between Part B 54 and Part C 55 when put or mated together Part C 55 has a male sample inlet (fitting) nipple 55a designed for a 5/16 inch inner diameter hose H2 to be affixed. The interior diameter of the (fitting) nipple 55a is 4.5 millimeters. The inlet canal 55e terminates in the mixing chamber 55c through a diffusion nozzle 55b. The diffusion nozzle 55b includes a plurality of spaced holes 55g1, 55g2, 55g3 and 55g4 which diffuse air therefrom into the mixing chamber 55c. The diffusion nozzle 55b is designed to create a turbulent entrance of air into the mixing chamber 55c and increase the probability of even deposition of particulates on the filter 56 during sampling periods. Part C 55 is designed to be compatible with existing technology of a standard 37 millimeter cassette and can be inserted on a standard 37 millimeter cassette when mixing for even deposition of particulates on the surface of the 37 millimeter filter is desired.

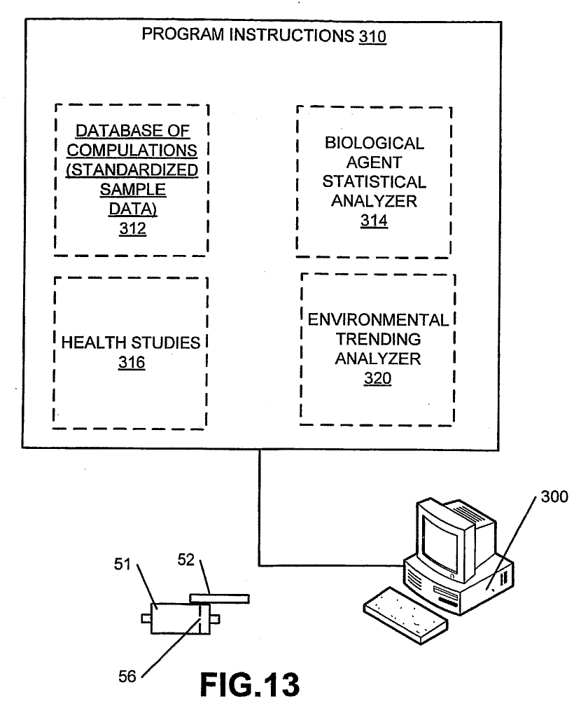

The data storage unit 52 communicates with either the microprocessor 152 of the main processor board PCB-1 when installed in the environmental sampling/sensor probe assembly 50 or with a computer 300 (FIG. 13) with a computer readable medium driver or card reader and appropriate software. Typically the laboratory would then send the downloaded data to the investigator (industrial hygienist) or company that conducted the sampling for reporting back to the habitants, building owner or manager.

The 4.5 millimeter opening in the male inlet 55a and outlet 53a, due to the possibility of contamination or spillage and thus the investigator will need to insure the male outlet (fitting) nipple 53a and male inlet (fitting) nipple 55a are sealed with plugs 53g and 55f (FIG. 4A) respectively, until ready for analysis at the lab. In a most preferred embodiment, the male outlet and inlet (fitting) nipples 53a and 55a are capped both prior to attaching or immediately after the removable cassette device 51 is removed from the attached pneumatic hoses H1 and H2.

Referring again to the particle counter 60, as best seen in FIGS. 1A, 1C and 4E, the peripheral particle counter 60, includes a counter housing 62 which can be mounted inline upstream of the removable cassette device 51, will receive power and communicate directly with the vacuum pump and system control assembly 20 through cable C7, splitter 25 (25′) and cable C8. The particle counter 60 will be attached to the inlet side of the removable cassette device 51 at inlet (fitting) nipple 55a with a 5/16 inch inner diameter hose H2.

In operation, the particle counter 60, a laser particle counter, counts some or all of the many particulates in the sample of air that enter the sampling port 60b. One or more particle counters 60 are designed to read particulates sizes of, but not limited to, 0.3, 0.5, 1, 2, 3, 5 and 10 microns. A particle counter 60 measuring 0.3 microns can determine effects of a Hepa Filter, if installed at a location. On the other hand, a particle counter 60 measuring 2-10 microns can detect molds or other biological particulates. This provides additional environmental data on the volume or number of air particulates entering the removable cassette device 51 and, thus, on the volume or number of air particulates in the sample air stream. The particle counter 60 is installed in a manner which allows it to be interchanged. The housing 62 of any particle counter 60 should include a connection port C2 for electrical integration into the system 10.

In the exemplary embodiment, depending on the suspected biological particulates, the investigator can change the particle counter 60 to accommodate the micron size of the suspected biological particulates at a location or for other diagnostic evaluation such as, without limitation, whether a Hepa Filter is functional.

The housing 62 includes a male outlet 60a, such as a male outlet barb, which attaches the particle counter 60 to a 5/16 inch inner diameter hose H2. The hose H2 connects to the male inlet (fitting) nipple 55a of the removable cassette device 51.

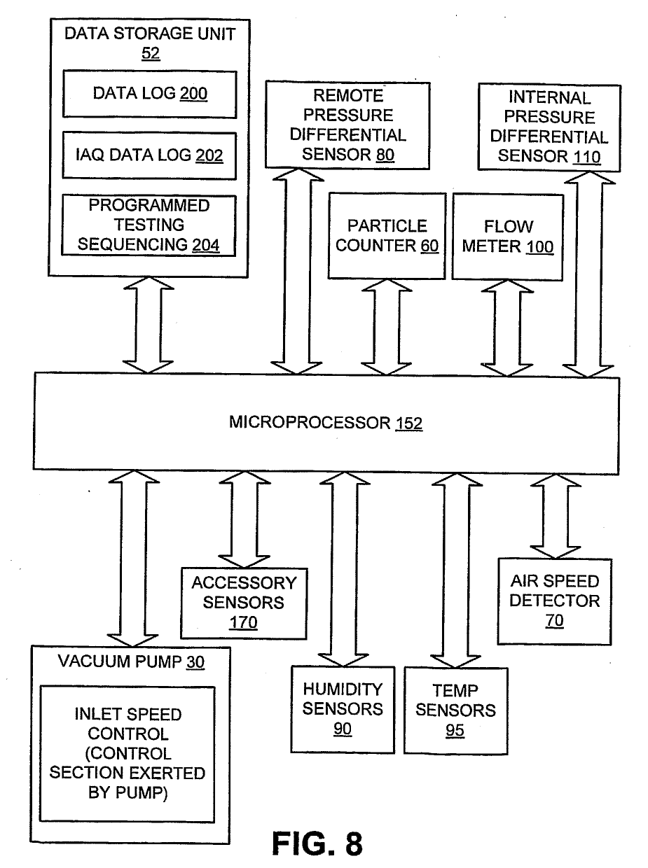

In FIG. 1A, the vacuum pump and system control assembly 20 includes an internal pressure differential detector 110 (FIG. 8) includes two male (fitting) nipples 20l and 20m attached to the housing 28. In the embodiment, of FIG. 1C, a remote pressure differential sensor 80 is provided. As best seen in FIG. 1C, the pressure differential sensor 80, includes housing 82 which has male (fitting) nipples 84a and 84b, such as two hose barbs, that can accommodate ¼ inch inner diameter pneumatic hoses (not shown). On at least one of the nipples 84a and 84b may be placed a hose where the other free end of the hose is placed in the location where the pressure differential is to be sensed. Multiple pressure differential sensors 80 (up to 32 boxes) can be used simultaneously with the environmental sampling system 10 expanding splitter 25′. The pressure differential sensor 80 will receive power and communicate directly with the vacuum pump and system control assembly 20 through the cable C10 attachments via port C11

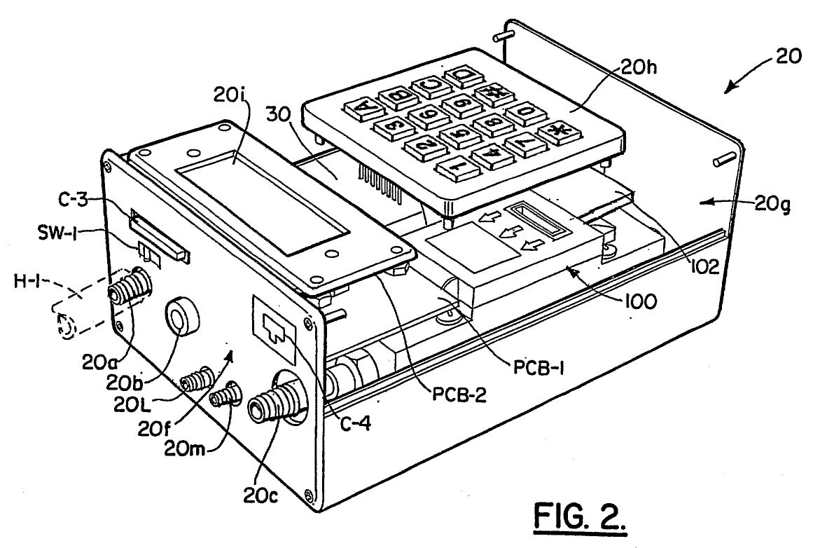

Referring again to FIG. 1A and FIG. 2, a 5/16 inch inner diameter hose H1 will connect the (e.g., Schwarzer Prazision SP 670 EC) of the vacuum pump and system control assembly 20 to the removable cassette device 51 via a male fitting 20a located on the top plate 20f of housing 28. This male (barbed) fitting 20a will be attached to the vacuum pump 30 integrated in the housing 28 of the vacuum pump and system control assembly 20 by appropriate pneumatic tubing. The exhaust of the vacuum pump 30 will be attached to the air flow meter 100 by appropriate pneumatic tubing. The exhaust of the air flow meter 100 will be attached to an exhaust male (barbed) fitting 20c in the top plate 20f.

The air flow meter 100 will monitor the air volume being drawn through the sample whenever the vacuum pump 30 is in operation and pulling air through the tubing (hoses H1, H2, H3) attached to the removable cassette device 51 or particle counter 60 and through sampling port 60b. This air flow information can be used to determine the air velocity at the inlet cone shape of the sampling port 60b to the laser particle counter 60, the removable cassette device's male inlet (fitting) nipple 55a, or an attached section of 5/16 inch inner diameter tubing (not shown) which feeds the removable cassette device 51. Thus, it can be used to regulate the force of the vacuum pump 30 so as to match the air speed in the sample collection hood (sample inlet opening of the laser particle counter, or removable cassette device or attached tubing) with equal or greater than the air speed in the HVAC duct cavity (as detected by the air speed detector 70).

The air speed detector 70 includes any off the shelf air speed detectors which can be inserted into the HVAC duct to determine the speed of air within the duct. The air speed detector 70 includes a handle section 73 with an elongated probe member 71. The tip of the probe member 71 has formed therein a slit 72 on two side of the probe member 71. In the slit 72 a wire 76 with a bead 74 is affixed in the tip of the probe member 71. In the exemplary embodiment, the probe member 71 should be thin enough to enter a slot in a duct register 3, as best seen in FIG. 6, serving as at least one part of system 10 being inserted or installed in the HVAC's system air duct 2.

As shown in FIGS. 1A, 1B, and 5, the air speed detector 70 receives power and communicates with the vacuum pump and system control assembly 20. The air speed detector 70 produces a standard variable signal that is interfaced such as via an A/D converter to the microprocessor 152 on the main printed circuit board PCB1.

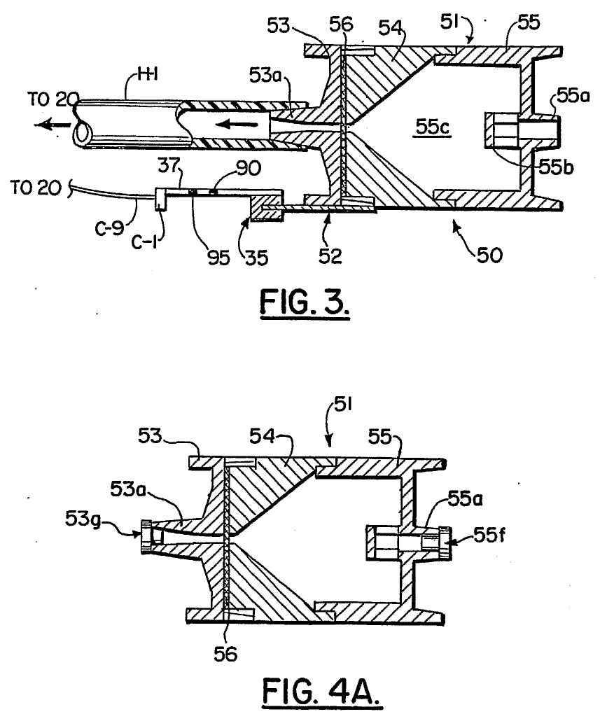

As shown in FIGS. 3 and 4E, the card holder 35 may include a sensor board 37 which includes a humidity sensor 90 and a temperature sensor 95 to detect the humidity and temperature of the sampled data such as in or near the air duct 2. Additionally, the housing 28 may include a humidity sensor and temperature sensor. Nevertheless, multiple humidity sensors 90 and temperature sensors 95 may be connected to the vacuum pump and system control assembly 20 via splitter 25 (25′) or other connectors on housing 28. The data of each sensor would be recorded. In this embodiment, the cable C9 is illustrated connected to the sensor board 37.

In the preferred embodiment, the humidity sensor 90 and a temperature sensor 95 should be removable, readily changed, and accessible. Moreover, the housing 28 would not have to be opened to access the humidity sensor 90 and a temperature sensor 95.

FIG. 6 illustrates the environmental sampling/sensor probe assembly 50 mounted in an air duct cavity of an HVAC system (a first installation mode). In the first installation mode, the environmental sampling/sensor probe assembly 50 is connected to the vacuum pump and system control assembly 20 with the pigtails of electrical wires (cables) and pneumatic tubing connecting the two assemblies. To install the environmental sampling/sensor probe assembly 50, a section of 5/16 inch inner diameter hose H3 is attached to either the removable cassette device male inlet (fitting) nipple 55a or the laser particle counter's sample port 60b. In the exemplary embodiment, one end of the hose H3 is attached to the particle counter 60. However, if a particle counter is not used in the sampling, then the hose H3 would be used in lieu of hose H2. The opposite end of the hose H3 is placed in the air stream of the HVAC air duct 2 adjacent to the location of the inserted air speed detector 70 which is attached via cables C6 to the vacuum pump and system control assembly 20. The hose H3 and air speed detector 70 may be attached to the air duct register 3 with wire clips, tape or other fasteners. Alternately, the hose H3 and air speed detector 70 could be held in place by a field technician.

When removing the removable cassette device 51, such as after the sampling period is complete, the particle counter 60 is disconnected by removing the attached flexible pneumatic tubing H2 and placing a plug 55f in the removable cassette device's male inlet (fitting) nipple 55a. Thereafter, the removable cassette device 51 can be disconnected from the vacuum pump and system control assembly 20 by removing the attached flexible pneumatic tubing H1 and placing a plug 53g in the removable cassette device's male suction (fitting) nipple 53a and disconnecting the data storage unit 52 by removing the card holder 35 from the data storage unit 52.

Referring now to FIGS. 1A, 1B and 2, the vacuum pump and system control assembly 20 includes housing 28 with an attached battery chamber 27 for housing a battery source 27 intended to power the components of both the vacuum pump and system control assembly 20, the environmental sampling/sensor probe assembly 50, the particle counter 60, the air speed detector 70 and pressure differential sensor 80. The vacuum pump 30 pulls air through the removable cassette device 51 and includes internal hoses (not shown). One of the internal hoses is in turn connected to at least one external hose H1 via (fitting) nipple 20a. The other of the internal hoses 102 is used to vent the air from the air flow meter 100 (alternatively, vents could be included in the housing for remote control assembly 20 to achieve venting).

The system 10 includes an adaptor connection 20b which provides DC power to the system 10. The attached battery source has a connector which allows an A/C to D/C adaptor to charge the system 10 or provide operating power to the system 10 or alternatively a plug to connect to a vehicle's cigarette lighter or other jack for recharging the battery source in battery chamber 27 or provide operating power to the system with the vehicle's battery (as used here, vehicle means any transport means such as car, bus, airplane, train, etc.). Moreover, if the system 10 is used to sample in a vehicle, the vehicle's battery can be used as the power source with the battery source being a back-up power source.

The vacuum pump and system control assembly 20 also includes a power on/off switch SW1.

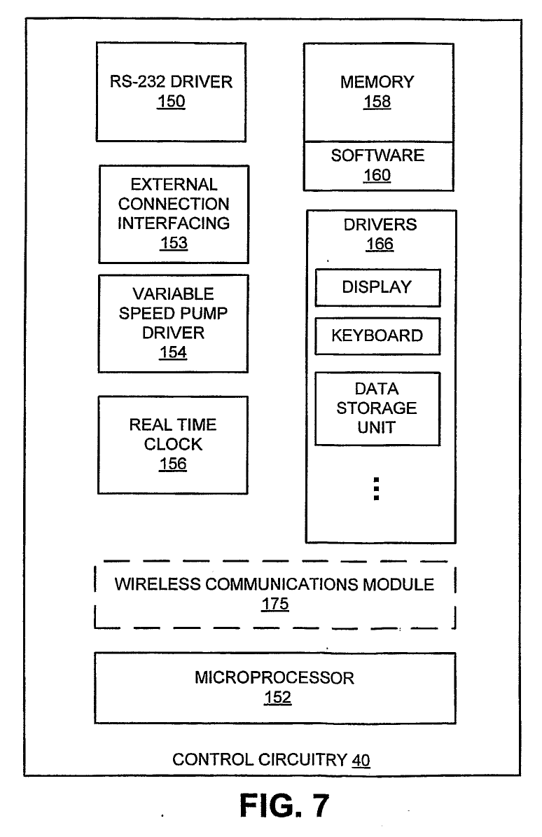

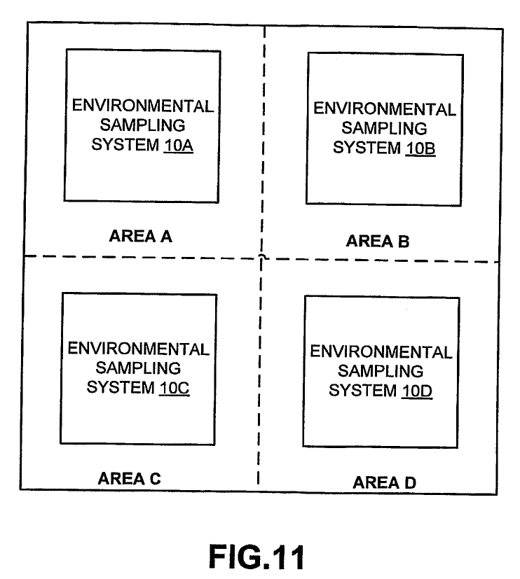

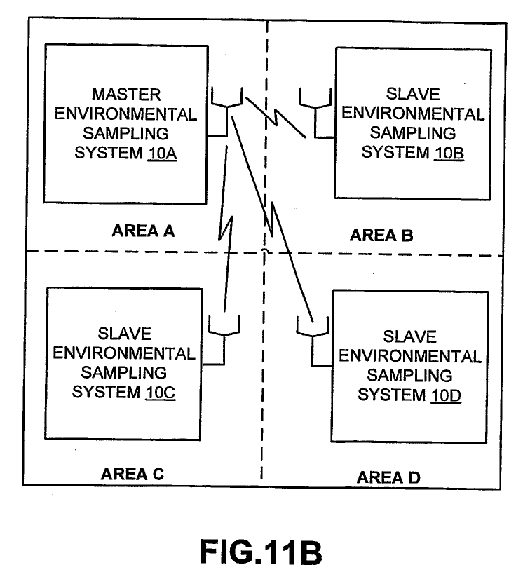

Referring also to FIGS. 2, 7 and 8, the vacuum pump and system control assembly 20 further includes a wireless communication module 175 or zigbee (e.g., Maxstream XBee) for communicating with accessories, other environmental sampling systems 10B, 10C, 10D (with one acting as the master and the others functioning as slaves, FIG. 11B) and control circuitry 40 integrated in the main printed circuit board PCB-1. The data base 200 could be logged on one data storage device 52 or multiple data storage devices if multiple systems 10A, 10B, 10C and 10D are used. However, only one storage device 52 with a synchronized time clock 156 is preferred.

The control circuitry includes a RS-232 driver 150, microprocessor 152, variable speed pump drive 154, real time clock 156, and other parts such as memory 158 and software 160 needed to carry out the functionality described herein. The control circuitry 40 also includes external connection interfacing 153 for connection to the connectors C3, C4, etc. and driver 166 for the display, keyboard, data storage unit transfer, accessory sensors, etc. The variable speed pump drive 154 varies the voltage to the vacuum pump's motor. The variable speed pump drive 154 is also interfaced with mass flow meter 100.

In the exemplary embodiment, the accessories for communicating via wireless communications may include another vacuum pump and system control assembly 20 and/or environmental sampling/sensor probe assembly 50 installed in the same building but in a different room. These accessories may include any one of accessory sensors 170 described below.

As shown in FIG. 2, a display 20i is provided in housing 28, such as a liquid crystal display (LCD), which is shown integrated with a printed circuit board PCB-2. Furthermore, an external keyboard 20h is mounted to housing 28, which communicate with the microprocessor 152 of the vacuum pump and system control assembly 20 to control the operations of the environmental sampling/sensor probe assembly 50, particle counter 60, air duct speed detector 70, pressure differential sensor 80, accessory sensors 170 and vacuum pump and system control assembly 20. The vacuum pump and system control assembly 20, its software 160 and keyboard 20h would allow the investigator (industrial hygienist) to choose one of several modes of operation, including, without limitation, the following: 1) elect sampling in an HVAC system for a cycle or to follow the HVAC cycle (see two paragraphs below); 2) sample for a sample period duration followed by a non-sampling period duration wherein the sample period duration and non-sampling period duration are repeated for a predetermined number of times or cycles and wherein the durations (number of minutes) for the sample period and the non-sampling period may be entered by user; and 3) sample in accordance with a particular sample standard for the application and mode of operation.

The software 160 is the core instruction set for operating the vacuum pump and system control assembly 20 and interfacing with the data storage unit 52 and programmed testing sequencing 204. The programmed testing sequencing 204 interfaces with, and is used by, the software 160 for carrying out the testing and sampling of indoor air, ambient air, and the HVAC system of a variety of building sciences.

When sampling in an HVAC system with the environmental sampling/sensor probe assembly 50 installed near an air duct 2, the system 10 may be selectively synchronized to perform sampling with the HVAC system cycling on and off periods. Thus, sampling may be programmed to take place when the HVAC system is circulating air in the air duct cavity. Alternately, in a second mode, the sampling may take place when the HVAC system is off or when no air is circulating in the interior cavity of the air duct 2. In FIG. 6, the housing 28 of the vacuum pump and system control assembly 20 is hooked to or suspended from the air duct register 3, via hooking or fastening members 29, if the air duct 2 is in a ceiling. However, if the register 3 is in installed in the floor, the housing 29 would be secured simply by placement on the floor.

In a preferred embodiment, a data input screen or graphical user interface would be provided to require the operator to input certain information before allowing the operator to operate the environmental sampling system 10. Such feature is a safety feature and ensures data integrity feature.

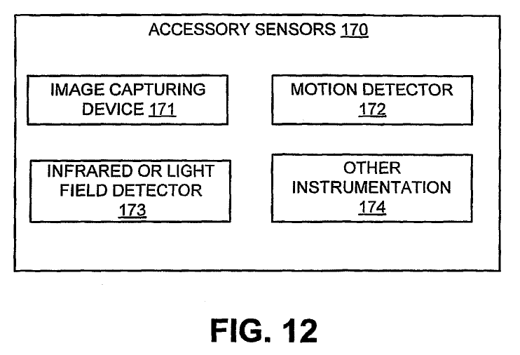

In FIG. 12, the accessory sensors include without limitation, (whether connected via wire or wireless to vacuum pump and system control assembly 20) motion detectors 172, image capturing device 171 such as a digital or video camera, actuator switches (as well as other switches), light field detectors 173 or other capture or instrumentation devices 174. In the exemplary embodiment, accessory sensors 170 may also include a laptop, an input device, an output device such as a printer, The motion detector 172 may detect movement at a location. The camera may determine human occupancy or when janitorial services are conducted at a location. The data of these additional sensors can be used to further correlate environmental effects at a given sample time period.

For example, the image capturing device 171 or motion detector 172 can be used to detect an event which causes sampling to take place or correlate sampling data with the event. When the janitor or maid service begins cleaning such as vacuuming and dusting, a change in the logged data in the data log 200 may be detected and/or correlated. Correlating events in a building or room to the logged data helps identify trends or a source of the contamination.

The system 10 is constructed and arranged to detect a cause of contamination which may require an accessory sensor not described herein since to describe each and every possible permutation is prohibitive. The sensors and sampling process is intended to detect events as simple as turning on and off a HVAC system as a source of contamination or a degraded Hepa filter. More complex events however may arise such as leaks after a rain can be detected by humidity sensors or leaks in the air duct could be evaluated by a lowering of temperature in the attic. The examples above are for illustrative purposes only and should not be considered exhaustive.

The cables C6, C7, C9 and C10 and hoses H1 and H2 may be twelve (12) feet or longer so that the vacuum pump and system control assembly 20 can be positioned at or near the floor and the environmental sampling/sensor probe assembly 50 installed in an air duct 2.

In general, as best seen in FIG. 8, the microprocessor 152 of the control circuitry 40 communicates with the data storage unit 52 and stores all data parameters thereon in data log 200. During sampling, the microprocessor 162, communicates with the vacuum pump 30, the air speed detector 70, the particle counter 60, and data store unit 52 to control the operations of system 10. The internal pressure differential detector 110, remote pressure differential detector 80 and air speed detector 70 are inputs to the microprocessor 152. Controls to the vacuum pump 30 are outputs of the microprocessor 152. The microprocessor 152 also communicates with any accessory sensors 170 connected to connectors C3, C4 or other connectors (not shown).

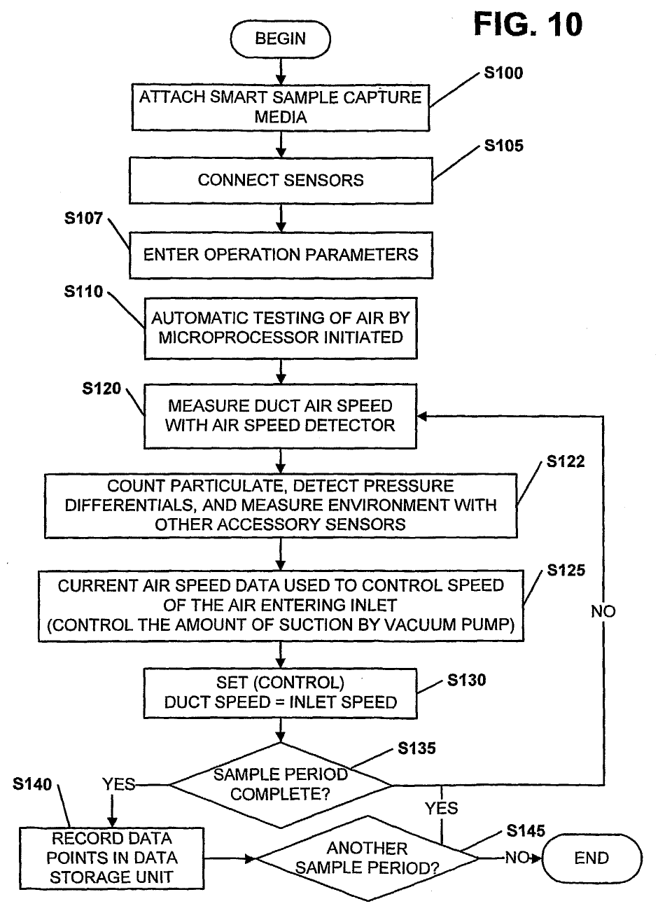

Referring now to FIG. 10, an exemplary flow chart of the sampling operations of system 10 is shown. The operations begin at step S100 with attaching the smart sample capture media which includes both the data storage unit 52 affixed to the removable cassette device 51 of the environmental sampling/sensor probe assembly 50 and installing the environmental sampling/sensor probe assembly 50. Step S100 is followed by step S105 where the sensors are connected such as particle counter 60, air speed detector 70, pressure differential detector 80, humidity sensor 90, and temperature sensor 95. Step S105 is followed by step S107 where the operator enters the operational parameters for conducting the sample such as, without limitation, sample duration, number of samples, if repeated, etc. and otherwise initializes the system 10. Step S107 is followed by step S110 where the microprocessor 152 initiates the testing or sampling. Step S110 is also followed by step S120 where the duct's air speed is measured by the air speed detector 70. Step S120 is followed by step S122 where the particle counter 60 counts particulates, pressure differentials are detected and other sensors (temperature sensor, humidity sensor, etc.) sense other parameters of the environment. Step S122 is followed by step S125 where the current air speed data is used to control the speed of the air entering the inlet port 60b by controlling the amount of suction by vacuum pump 30. Step S125 is followed by step S130 where the inlet speed is set to the duct speed. Step S130 is followed by step S135 where a determination is made whether the sample duration is complete. If the sample duration is complete, the data is stored into database log 200 in the data storage unit 52, at step S140. Step S140 is followed by step S145 where a determination is made whether another sample period is to be conducted. If the determination is “NO,” operations are ended. Otherwise, if the determination is “YES,” the process returns to step S120.

Also if the determination at step S135 is “NO,” the process loops back to step S120 to continue with the sampling. Alternatively, the data can be logged into database log 200 in the data storage unit 52 throughout, or at periodic times or events, during the sampling (and thus before the sampling is completed).

In some applications, the entire environmental sampling/sensor probe assembly 50 is installed in a large duct. Thereby, the vacuum pump and system control assembly 20 and environmental sampling/sensor probe assembly 50 may remain together and installed in a large duct (the second installation mode). In a third installation mode, the entire system 10 as seen in FIG. 6 will be placed near the HVAC register (return or supply) but only a pneumatic tube (a collection hose) and air speed detector is run into the HVAC system for collecting a sample. While, the environmental sampling/sensor probe assembly 50 is described to sample air in an air duct of an HVAC system, the sampling/sensor probe assembly 50 can sample ambient air indoors or outdoors.

The top plate of the housing 28 is attached to the top and bottom housing sections with four flat head screws. The back plate 20g of housing 27 is also attached to the top and bottom housing sections with four flat head screws. The cables and pneumatic tubing is removed for transportation of the unit. By using short sections of thick walled pneumatic tubing H1 and a cable C9 to connect C1 to C4, the environmental sampling/sensor probe assembly 50 can be mounted directly on top of vacuum pump and system control assembly 20. This configuration holds the vacuum pump and system control assembly 20 and the environmental sampling/sensor probe assembly 50 together for mounting inside of a larger duct or for transportation of the system 10. An AC-to-DC converter and battery recharger is incorporated into vacuum pump and system control assembly 20 for those environments where reliable power (whether AC or DC) is available (with the battery then serving as an emergency backup power source).

In the preferred embodiment, the environmental sampling/sensor probe assembly 50 should be installed substantially in the middle of the air-duct cavity (as greater air speed, air volume and air particulates are generally in the center of the air stream).

### Modes of Operation

The environmental sampling system 10 can be used in four modes of applications related to, but not limited to, commercial, industrial, transport vehicles, residential structures or for outdoor environmental evaluation. Each mode will be described below.

### Mode 1: Air-Duct Sampling

When checking a HVAC system, the entire environmental sampling system 10 is capable of being installed inside the HVAC system ductwork cavities, or with portions of the environmental sampling system 10 (vacuum pump and system control assembly 20 and the environmental sample/sensor probe assembly 50) being set up outside the HVAC system and a sampling probe (flexible tubing) with the air speed detector 70 being placed inside the HVAC system ductwork cavities. This environmental sampling/sensor probe assembly 50 is designed to collect environmental samples from both the intake and exhaust side (supply and return locations) of a HVAC system. The purpose for collecting this environmental data in sample data log 200 is to obtain information on the HVAC system's performance, check for amplification of biological agents caused by the HVAC system and to check for the transmission or distribution of microbial or other particulates through the HVAC system from other locations such as a contaminated room within a structure or from sources external to the structure. To assist the investigator, the environmental sampling system 10 is designed to data log all the physical environmental data on the data storage unit 52 of the removable cassette device 51 which data will be downloaded by the analytical laboratory and included as part of the report from the analytical laboratory back to the investigator with the analysis of the particulates information obtained from the filter media in the removable cassette device 51. The real time data logging of the particulates concentrations that pass through the sampling/sensor probe assembly 50 with a laser particle counter 60 and with the biological loading on the filter media will give indications of any cycles of biological concentrations which may have occurred during the sampling period. The data logging of the sample time and sample flow rates will allow the standardization of samples for comparison. The samples can be analyzed with statistical means to differentiate between different populations of biological agents and with differentiation between populations. Epidemiological studies will allow evaluation of indoor concentrations of biological growth as they relate to illness. After sufficient data is collected to establish relative cleanliness levels of HVAC systems regionally, interpretations will be possible to determine the potential for the HVAC system to negatively impact the indoor air quality of the residential, commercial or industrial structures or transport vehicles. The physical data obtained will give a strong indication of the overall performance of the structures/vehicle as it relates to indoor air quality and moisture intrusion via humidity issues. The long term goal is to establish a database with significantly better information, than is presently available to epidemiologists, which can be used in further health related studies and in the development of better building practices.

Referring now to FIG. 9A, the environmental sampling system 10 can evaluate and data log in sample data log 200, the operation of a Heating Ventilation and Air Conditioning system relative to: 1) biological contamination including, but not limited to, airborne particulate levels of organisms such as fungi, bacteria, dust mites, pet dander and viruses via the filter in the removable cassette device 51; 2) equipment operations and environmental physical parameters including, but not limited to, temperature, relative humidity, particle density, and micro-environmental pressure differentials; and, 3) information on the operation of the environmental sampling system 10 such as start time, stop time, time of year, air flow levels in the duct work and within the environmental sampling system 10.

### Mode 2: Sampling Ambient Air

In this mode the environmental sampling system 10 would be set to run at a predetermined on-off rate of air flow on continuous run or to sample at predetermined on-off intervals, for a set amount of time. The sampling would also log on the data storage unit 52 in real time (through use of the microprocessor 152 all of the other data collection devices (pressure differential monitors, laser particle counter 60, and temperature and humidity readings and air speed/air flow levels, time of day, or time of year reading, etc.). The logging of all collected data from the investigation on to a data storage unit 52, logged in, in real time, means such data is attached to the removable cassette device 51 and sent to the laboratory so that it can be reported back with the laboratory sample results. The data will also be compiled into a larger database so that further scientific investigation of many samples can be compared in order to detect trends and other similar characteristics among samples. The goal is to allow for the database to be used in further health related studies and in the development of better building practices.

The environmental sampling system 10 evaluates the indoor or outdoor air quality of any ambient air relative to: 1) biological contamination including, but not limited to, airborne particulate levels of organisms such as fungi, bacteria, dust mites, pet dander and viruses; 2) equipment operations and environmental physical parameters including, but not limited to, temperature, relative humidity, particle density, and micro-environmental pressure differentials; and, 3) information on the operation of the environmental sampling system 10 such as start time, stop time, time of year, and air flow levels within the sampling device.

Mode 3: Building Data Logging without Air Sampling Laboratory Report

In this mode the data from one or more or all of the sensors (laser particle counter 60, pressure differential sensor 80, temperature and humidity) as well as air speed, air flow, time of day, and time of year would all be logged to the data storage unit 52 but the laboratory analysis would not be completed at the initial reporting time. This would allow the data to be examined at the time of the investigation. The collection of air flow information, particle counts, the logging of all collected data from the investigation on to a data storage unit 52, logged in real time that will be attached to the removable cassette device 51 and sent to the laboratory so that it can be reported back with any other laboratory sample results. For example, a room or area of a location can be monitored for equipment operations and environmental physical parameters without the need to evaluate, by the laboratory, the biological particulates or microbial data captured by the sample capture media. However, other rooms of the location may need to evaluate biological particulates or microbial data. Therefore, laboratory sample results for the location would then contain the data stored on all the data storage units 52 of the different rooms or areas but only some of the biological particulates or microbial data from some of the sample capture media as specified by the investigator or company performing the sampling.

The data will also be compiled into a larger database so that further scientific investigation of many samples can be compared in order to detect trends and other similar characteristics among samples. The goal is to allow for the database to be used in further health related studies and in the development of better building practices.

The environmental sampling system 10 logs data from other building diagnostic investigation relative to operations and environmental physical parameters such as temperature, relative humidity, particle density, and micro environmental pressure differentials.

### Mode 4: Sample Pump

The sampling device is capable of using many types of sample capture media such as filter cassettes, slit impaction units, traditional impactors, impingers, or traditional industrial hygiene sampling tubes where the intake air time and flow (volume) can be monitored, measured and all of the information can be recorded on the data storage unit 52. The collection of air flow information, particle counts, the logging of all collected data from the investigation on to a memory chip, logged in real time, is attached to the sample capture media and sent to the laboratory so that it can be reported back with the laboratory sample results. The data will also be compiled into a larger database so that further scientific investigation of many samples can be compared in order to detect trends and other similar characteristics among samples. The goal is to allow for the database to be used in further health related studies and in the development of better building practices.

As a sampling pump using existing sample media such as filter cassettes, sample tubes, slit impactors, traditional impactors and impingers, the environmental sampling system 10 will be able to record all environmental parameters relative to the sampling event and recorded on the laboratory report after analysis.

Referring now to FIG. 11A, a location with multiple areas A, B, C and D is shown. Each area A, B, C and D has at least an environmental sampling system 10A, 10B, 10C and 10D with at least an environmental sampling probe assembly 50, installed to sample the environment. The laboratory would evaluate the biological particulates or microbial data on the sample capture media and download the data from the data storage unit 52 associated with each environmental sampling/sensor probe assembly 50. Such data is then sent to the investigator or company performing the sampling to report to the building owner or manager, homeowner or other habitants the total building profile or analysis. For example, in a grocery store, the data from area A may indicate the presence of certain bacteria from meat. On the other hand, the data from area B might indicate elevated moisture content in the air. The data collectively from all areas may indicate an elevation of particulates, when the HVAC is turned on, and other biological contamination. Thus, the total building profile can be used to evaluate and diagnose equipment operations to improve the environment.

In FIG. 11B, the system 10A is a master environmental sampling system with the systems 10B, 10C and 10D as slaves. The master system 10A is in wireless communications with the slaves systems 10B, 10C and 10D. In this embodiment, the data storage unit 52 of the master may be the only means of storing the data with a synchronized time clock.

Referring now to FIG. 9B, the sample data log 200′ may includes data entries for a plurality of particle counters 60¹, 60², . . . 60^{x}, a plurality of air speed detectors 70¹, 70², . . . 70^{x}, a plurality of humidity sensors 90¹, 90², . . . 90^{x}, a plurality of temper sensors 95¹, 95², . . . , 95^{x} and pressure differential detectors 80¹, 80², . . . 80^{x}. The multiple entries may come from different systems 10A, 10B, 10C, 10D or multiple sensors attached to a single system 10.

Referring now to FIG. 13, at the laboratory a computer 300, such as a personal computer (PC), laptop or other computing device having an operating system thereon would be used to download the sample data log 200 or 200′. After the data log 200 or 200′ and the IAQ data log 202 are downloaded to computer 300, an analysis of the data would take place. For example, the data would be compiled in a manner to develop a standardized sample data with programming instructions 312. Then biological agent statistical analyzer 314 could be used to evaluate the data in the filter 56. The health studies 316 would collect the IAQ data log 202 with medical parameters and conditions and evaluate to create trends in the environmental trending analyzer 320. For example, a trend may correlate an event, such as dusting, turning on the HVAC system, rain, etc. to a rise in biological agents causing a correlated health effect.

In view of the foregoing, the present invention provides an environmental sampling system for air particulates comprising: a means for sampling the air to create an air sample and a means for capturing from the air sample. The sample of the air particulates in the air sample is captured by a removable cassette device 51 which has an inlet diffusion nozzle in communication with a mixing chamber 55c. The mixing chamber 55c, having a minimum length of 15 millimeters to a maximum length of 100 millimeters, promotes the even distribution of particulates on the capture filter 56. The filter 56 has a 37 millimeters diameter or the capture filter 56 may be reduced down to a 2 millimeter by 22 millimeter trace with a funnel 54a causing the particulates to be concentrated evenly on the filter trace from the mixing chamber 55c, with raised 1 millimeter pins 54b to mark the 37 millimeter filter 56 at the quarter and half lengths of the trace allowing the analytical laboratory to separate the trace into equidistant sections for various analysis.

A means for storing (data storage unit 52) the air particulates sample in a removable cassette device 51, such as without limitation a memory card, may be provided.

The mixing chamber 55c provides mixing of the particulates in the air in a manner as to provide a statistically demonstrated even distribution on the sample filter allowing analysis of a portion of the filter 56 as being a representative sample of the entire filter particulate population. A plurality of sensors may be provided for sampling a plurality of environmental physical parameters associated with the air sample during or surrounding a sampling period during which the air sample was sampled.

Furthermore, the removable cassette device 51 has a filter 66 or other media for capturing and storing the air particulates sample, including biological particulates, and a data storage unit 52 affixed thereto for storing samples of said plurality of environmental physical parameters and wherein, after the sampling period, the removable cassette device 51 is sent to a laboratory so that both the air particulates sample, which may contain biological contamination, in the filter 56 in the removable cassette device 51 can be determined and the stored environmental physical parameters and IAQ questionnaire information can be downloaded from the data storage unit 52 for evaluation with the air particulates sample.

The sample capture media may include one of filter cassettes, slit impaction units, traditional impactors, impingers, or traditional industrial hygiene sampling tubes where the intake air time and flow (volume) can be monitored, measured and all of the information can be recorded on the data storage unit 52.

The present invention provides a method of checking a HVAC system comprising: installing the environmental sampling system or environmental sampling/sensor probe assembly 50 inside the HVAC system ductwork cavities, or with portions of the environmental sampling system (vacuum pump and system control assembly 20) being set up outside the HVAC system and a sampling probe with media (environmental sampling/sensor probe assembly 50) being placed inside the HVAC system ductwork cavities. The method includes collecting by the environmental sampling/sensor probe assembly 50 environmental samples from both the intake and exhaust side (supply and return locations) of the HVAC system and logging, during sampling, all the physical environmental data on the data storage unit 52 of a removable cassette device 51 which is adapted to be downloaded by an analytical laboratory or the investigator. The method includes obtaining particulates information from a sample capture media in the removable cassette device 51 and real time data logging of the particulates concentrations that pass through the cassette device 51 with a peripheral laser particle counter 60 and with the biological loading on the sample capture media will give indications of any cycles of biological concentrations which may have occurred during the sampling period. The method also includes logging of the sample time and sample flow rates for standardization of samples for comparison and analyzing the samples with statistical means to differentiate between different populations of biological agents and with differentiation between populations, wherein epidemiological studies will allow evaluation of indoor concentrations of biological growth as they relate to illness.

The collecting step may include: obtaining information on the HVAC system's performance, determining amplification of biological agents caused by the HVAC system; and, determining transmission or distribution of microbial or other particulates through the HVAC system from other locations such as a contaminated room within a structure or from sources external of the structure.

The method may further comprise the steps of: after sufficient data is collected to establish relative cleanliness levels of HVAC systems regionally, interpreting said sufficient data to determine the potential for the HVAC system to negatively impact the structure's indoor air quality and wherein the physical data obtained is adapted to provide a strong indication of a structure's overall performance as it relates to indoor air quality and moisture intrusion via humidity issues.

The method may further comprise the steps of: developing a database of said sufficient data for use by an epidemiologist or others; and, determining by an epidemiologist or others causality of health issues or diseases and to develop better building practices.

The method of sampling ambient air comprising the steps of: sampling using the environmental sampling system 10 at a predetermined rate of air flow on continuous run or to sample at predetermined on-off intervals, for a set amount of time to develop sample data; logging on a data storage unit 52 in real time all of the sample data from pressure differential sensor (110 or 80), laser particle counter 60, temperature sensor 95 and humidity sensor 90; and, collecting biological contamination in a removable cassette device 51 having the data storage unit 52 affixed thereto.

The method may further comprise the steps of: compiling into a larger database data related to the collected biological contamination and the stored sample data so that further scientific investigation of many samples can be compared in order to detect trends and other similar characteristics among samples as well as for use in further health related studies and in the development of better building practices.

The method may employ a sample capture media which includes one of filter cassettes, slit impaction units, traditional impactors, impingers, or traditional industrial hygiene sampling tubes where the intake air time and flow (volume) can be monitored, measured and all of the information can be recorded on the data storage unit.

The present invention provides a system for checking a HVAC system comprising: an environmental sampling system 10 having at least a part adapted to be installed or inserted inside a HVAC system's ductwork cavity, a sampling probe or capture media being placed inside the HVAC system's ductwork cavities; means for collecting by the environmental sampling/sensor probe assembly environmental samples from both the intake and exhaust side (supply and return locations) of the HVAC system; means for logging during sampling all the physical environmental data on a data storage unit of a removable cassette device which is adapted to be downloaded by an analytical laboratory or investigator; means for obtaining particulates information from a sample capture media in the removable cassette device; means for real time data logging of the particulates concentrations that pass through the cartridge device with a peripheral laser particle counter and with the biological loading on the sample capture media will give indications of any cycles of biological concentrations which may have occurred during the sampling period; means for logging of the sample time and sample flow rates for standardization of samples for comparison; and, means for analyzing the samples with statistical means to differentiate between different populations of biological agents and with differentiation between populations, wherein epidemiological studies will allow evaluation of indoor concentrations of biological growth as they relate to illness.

The collecting means includes: means for obtaining information on the HVAC system's performance; means for determining amplification of biological agents caused by the HVAC system; and, means for determining transmission or distribution of microbial or other particulates through the HVAC system from other locations such as a contaminated room within a structure or from sources external to the structure.

The system further comprising: means after sufficient data is collected to establish relative cleanliness levels of HVAC systems regionally; and means for interpreting said sufficient data to determine the potential for the HVAC system to negatively impact the structures indoor air quality and wherein the physical data obtained is adapted to provide a strong indication of a structure's overall performance as it relates to indoor air quality and moisture intrusion via humidity issues.

The system further comprising: a database of said sufficient data for use by an epidemiologist or others; and, means for determining by an epidemiologist or others causality of health issues or diseases and to develop better building practices.

The system for sampling ambient air comprising: means for sampling using the environmental sampling system at a predetermined rate of air flow on continuous run or to sample at predetermined on-off intervals, for a set amount of time to develop sample data; means for logging on a data storage unit in real time all of the sample data from pressure differential monitors, laser particle counter, temperature sensor and humidity sensors or other devices; and, means for collecting biological contamination in a removable cassette device having the data storage unit affixed thereto.

The system further comprising: means for compiling into a larger database data related to the collected biological contamination and the stored sample data so that further scientific investigation of many samples can be compared in order to detect trends and other similar characteristics among samples as well as for use in further health related studies and in the development of better building practices.

The system used without the sample capture media to record physical environmental data on the data storage device (such physical environmental data as, temperature, relative humidity, pressure differentials, wind velocities and particulate counts as they relate to evaluation of the tightness and operations of HVAC systems or infiltration rates, moisture intrusion and air leakage locations of buildings in general).

The system may include multiple environmental sampling systems which can be linked with wireless communications with one environmental sampling system functioning as the master control unit and the other environmental sampling systems functioning as slaves and providing simultaneously sampled areas A, B, C, D with all data being completely time correlated and said data being logged on one data storage device or multiple data storage devices.

Many varying and differing embodiments may be made within the scope of the inventive concept herein taught and because many modifications may be made in the embodiment herein detailed in accordance with the descriptive requirement of the law, it is to be understood that the details herein are to be interpreted as illustrative and not in a limiting sense.

What is claimed as invention is:

1. An environmental sampling system of air for air particles comprising:
means for suctioning the air to create an air sample;
means for sampling, in real time, a plurality of environmental physical parameters associated with the air sample;
means, removably coupled to the suctioning means, for capturing particles of the air sample and storing computer readable information related to the sampled plurality of environmental physical parameters; and,
means for controlling the suctioning means during a sampling period and writing the computer readable information related to the sampled plurality of environmental physical parameters to the capturing and storing means.

2. The system of claim 1, wherein the sampling means comprises:
means for counting particles from the air sample; and,
means for detecting air speed of the air sample.

3. The system of claim 2, wherein the sampling means further comprises:
means for sensing humidity in the air sample; and,
means for sensing a temperature of the air sample.

4. The system of claim 2, wherein the sampling means further comprises:
means for detecting a pressure differential in locations associated with the air sample; and,
means for sensing a temperature of the air sample.

5. The system of claim 1, wherein controlling means further comprises:
means for powering the sampling means and the capturing and storing means.

6. The system of claim 1, wherein controlling means further comprises:
means for communicating wireless with at least one a slave environmental sampling system.

7. The system of claim 1, wherein the capturing and storing means comprises: means for filtering particulates; and means for mixing of the particulates in the air sample in a manner as to provide a statistically demonstrated even distribution on a filtering means.

8. A method of sampling ambient indoor air comprising the method steps of:
sampling the air using the environmental sampling system at a predetermined rate of air flow for a set amount of time to develop sample data of a plurality of environmental physical parameters;
logging on a data storage unit in real time the sample data;
during the sampling, suctioning and collecting biological contamination of the sampled air in a removable cassette device, coupled to the environmental sampling system, the cassette device having the data storage unit affixed thereto.

9. The method of claim 8, wherein the sampling step comprises the steps of:
counting particles from the sampled air being suctioned into removable cassette device; and,
detecting air speed of the air.

10. The method of claim 9, wherein the sampling step further comprises the steps of:
sensing humidity in the sampled air; and,
sensing a temperature of the sampled air.

11. The method of claim 9, wherein the sampling step further comprises the steps of:
controlling suctioning of the suctioning and collecting step through the removable cassette device via the environmental sampling system.

12. The method of claim 9, wherein the sampling step further comprises the steps of:
detecting a pressure differential in locations associated with the sampled air during the; and,
sensing a temperature of the sampled air.

13. The method of claim 8, wherein the sampling step further comprises the steps of:
turning on a heating ventilation air conditioning (HVAC) system; and
wherein the sampled air is from an air duct cavity of the HVAC system.

14. The method of claim 8, further comprising the steps of:
receiving and logging information related to health conditions of persons in contact with the air being sampled in the data storage unit.

15. The method of claim 8, further comprising the steps of:
compiling, into a larger database, data related to the collected biological contamination and the stored sample data;
evaluating scientifically the compiled data, to detect trends and other similar characteristics among the sampled air.

16. The method of claim 8, further comprising the steps of:
detecting an event causing an amplification the biological contamination.

17. The method of claim 8, wherein the suctioning and collecting step comprises the steps of: filtering particulates of the sampled air; and mixing of the particulates in the sample air in a manner as to provide a statistically demonstrated even distribution.

18. A system for checking a heating ventilation air conditioning (HVAC) system comprising:
a sampling probe being placed inside an air duct of the HVAC system;
a control assembly with vacuum pump operable to suction an air sample through the sampling probe during a sampling period;
a plurality sensors, coupled to the control assembly, operable to sense environmental physical parameters;
data storage unit, electrically coupled to the control assembly, operable to have logged therein during the sampling period the sensed environmental physical parameters;
a removable cartridge device, affixed to the data storage unit, having a filter media operable to capture and collect biological contamination from the suctioned air sample.

19. The system of claim 18, wherein the plurality of sensors comprise:
means for real time data logging of the particulate concentrations that will be captured by the filter media; and,
means for logging of the sample time and sample flow rates.

20. The system of claim 19, wherein the plurality of sensors comprise:
means for detecting a pressure differential in locations associated with the air sample; and,
means for sensing a temperature of the air sample.

21. The system of claim 18, wherein control assembly further comprises:
means for powering the plurality of sensors.

22. The system of claim 18, wherein control assembly further comprises:
means for communicating wireless with at least one slave control assembly at a remote location.

23. The system of claim 18, wherein the system is operable to obtain information on the HVAC system's performance; determine amplification of biological agents caused by the HVAC system; and, determine transmission or distribution of microbial or other particles through the HVAC system from other locations.

24. The system of claim 18, wherein the removable cartridge device comprises: a filter media; a diffusion inlet operable to diffuse the sample air and a mixing chamber operable to receive the diffused air and mix of particulates in the diffused air in a manner as to provide a statistically demonstrated even distribution on the filter media.

25. A system for sampling ambient air comprising:
a plurality of linked environmental sampling systems, each system being associated with a corresponding location, and wherein each system comprises:
means for sensing a plurality of environmental parameters;
means for automatically controlling the sensing means for sampling the air at a predetermined rate of air flow on continuous run or to sample at predetermined on-off intervals, for a set amount of time to develop sample data from said sensing means;
means for logging and storing in real time the sample data; and,
means for collecting and loading biological contamination of suctioned sampled air, under control of the controlling means, having the logging means affixed thereto.

26. The system of claim 24, wherein the system is operable to obtain information on the HVAC system's performance; determine amplification of biological agents caused by the HVAC system; and, determine transmission or distribution of microbial or other particles through the HVAC system from other locations.

27. The system of claim 24, wherein one of the controlling means is a master controller and one of the logging means includes the sample data of all the plurality of linked environmental sampling systems.

28. A collecting and loading biological contamination device comprising:
a body having means for placing a storage data unit thereon and an outlet;
an inlet port, coupled to said body, operable to receive an air sample;
a interior chamber in communication with the inlet port; and,
a capture filter media, in said chamber and adjacent said outlet, operable to load the biological contamination in the air sample.

29. The device of claim 27, wherein the interior chamber is a mixing chamber.

30. The device of claim 28, wherein the mixing chamber has a minimum length of 15 millimeters to a maximum length of 100 millimeters and promotes an even distribution of particulates on the capture filter media.

31. The device of claim 29, wherein the capture filter media is 37 millimeters diameter and is adapted to be reduced down to a 2 millimeter by 22 millimeter trace.

32. The device of claim 28, wherein the outlet comprises a funnel causing particulates to be concentrated evenly on the capture filter media from the mixing chamber.

33. The device of claim 28, further comprising a plurality of pins operable to mark said capture filter media.

34. The device of claim 32, wherein each pin is a 1 millimeter pin to mark the capture filter media at the quarter and half lengths to allowing the capture filter media to be separated into equidistant sections.

35. A smart collecting loading biological contamination device comprising:
a computer readable medium operable to store and log therein computer readable data; and,
a body having means for placing the computer readable medium thereon, the body further comprising:
an outlet,
an inlet port operable to receive an air sample,
an interior chamber in communication with the inlet port, and
a capture filter media, in said chamber and adjacent said outlet, operable to load the biological contamination in the air sample wherein the body is constructed and arranged to be opened to retrieve the capture filter media and the stored data being downloaded.

36. The device of claim 34, wherein the chamber is a mixing chamber.

37. The device of claim 35, wherein the mixing chamber has a minimum length of 15 millimeters to a maximum length of 100 millimeters and promotes an even distribution of particulates on the capture filter media.

38. The device of claim 36, wherein the capture filter media is 37 millimeters diameter and is adapted to be reduced down to a 2 millimeter by 22 millimeter trace.

39. The device of claim 34, wherein the outlet comprises a funnel causing particulates to be concentrated evenly on the capture filter media from the mixing chamber.

40. The device of claim 34, further comprising a plurality of pins in said body operable to mark said capture filter media.

41. The device of claim 39, wherein each pin is a 1 millimeter pin to mark the capture filter media at the quarter and half lengths to allowing the capture filter media to be separated into equidistant sections.

42. The device of claim 34, wherein the computer readable medium is has stored therein programmed testing sequencing for conducting a test to capture the biological contamination in the air sample and other environmental parameters.

43. The system of claim 1, wherein the capturing and storing means has stored therein programmed testing sequencing for conducting a test to capture the environmental physical parameters and the particles of the air sample.

44. The system of claim 42, wherein the programmed testing sequencing is related to building science.

45. The system of claim 18, wherein data storage unit has stored therein programmed testing sequencing for conducting a test to sense the environmental physical parameters and collect the biological contamination from the suctioned air sample.

46. The system of claim 44, wherein the programmed testing sequencing is related to building science.

47. The system of claim 24, wherein the logging and storing means has stored therein programmed testing sequencing for conducting a test to sense the sample data and collect the biological contamination from the suctioned sampled air.

48. The system of claim 46, wherein the programmed testing sequencing is related to building science.

49. The method of claim 8, further comprising the step of:
executing programmed testing sequencing stored in the data storage unit, to perform the sampling step.
